SEQUENCE LISTING (146 sequences)

SEQ ID NO: 1 - DNA, 58 bp, Artificial Sequence
  Synthetic construct
aatgatacggcgaccaccgagatctacactctttccctacacgacgctcttccgatct

SEQ ID NO: 2 - DNA, 25 bp, Artificial Sequence
  Synthetic construct
aatgatacggcgaccaccgagatct

SEQ ID NO: 3 - DNA, 64 bp, Artificial Sequence
  Synthetic construct
aatgatacggcgaccaccgagatctnnnnnnacactctttccctacacgacgctcttccgatct

SEQ ID NO: 4 - DNA, 58 bp, Artificial Sequence
  Synthetic construct
caagcagaagacggcatacgagatgtgactggagttcagacgtgtgctcttccgatct

SEQ ID NO: 5 - DNA, 24 bp, Artificial Sequence
  Synthetic construct
caagcagaagacggcatacgagat

SEQ ID NO: 6 - DNA, 64 bp, Artificial Sequence
  Synthetic construct
caagcagaagacggcatacgagatnnnnnngtgactggagttcagacgtgtgctcttccgatct

SEQ ID NO: 7 - DNA, 33 bp, Artificial Sequence
  Synthetic construct
acactctttccctacacgacgctcttccgatct

SEQ ID NO: 8 - DNA, 23 bp, Artificial Sequence
  Synthetic construct
tcagacgtgtgctcttccgatct

SEQ ID NO: 9 - DNA, 38 bp, Artificial Sequence
  Synthetic construct
tcagacgtgtgctcttccgatctggaacgcacggacat

SEQ ID NO: 10 - DNA, 36 bp, Artificial Sequence
  Synthetic construct
tcagacgtgtgctcttccgatctcaagctgggcggg

SEQ ID NO: 11 - DNA, 40 bp, Artificial Sequence
  Synthetic construct
tcagacgtgtgctcttccgatctgaggaggaagtagcgtg

SEQ ID NO: 12 - DNA, 40 bp, Artificial Sequence
  Synthetic construct
tcagacgtgtgctcttccgatctcacccaaaagtccacct

SEQ ID NO: 13 - DNA, 45 bp, Artificial Sequence
  Synthetic construct
tcagacgtgtgctcttccgatctcagtgaaagattcatctaatgg

SEQ ID NO: 14 - DNA, 43 bp, Artificial Sequence
  Synthetic construct
tcagacgtgtgctcttccgatctcagacaactgttcaaactga

SEQ ID NO: 15 - DNA, 42 bp, Artificial Sequence
  Synthetic construct
tcagacgtgtgctcttccgatctaccttcaatgtgtttggtt

SEQ ID NO: 16 - DNA, 38 bp, Artificial Sequence
  Synthetic construct
tcagacgtgtgctcttccgatctggtaccgtgcgacat

SEQ ID NO: 17 - DNA, 39 bp, Artificial Sequence
  Synthetic construct
tcagacgtgtgctcttccgatctcctgtcgtcaactcct

SEQ ID NO: 18 - DNA, 42 bp, Artificial Sequence
  Synthetic construct
tcagacgtgtgctcttccgatctcagtcttacctggactctg

SEQ ID NO: 19 - DNA, 41 bp, Artificial Sequence
  Synthetic construct
tcagacgtgtgctcttccgatctgcagcatgtcaagatcac

SEQ ID NO: 20 - DNA, 44 bp, Artificial Sequence
  Synthetic construct
tcagacgtgtgctcttccgatctgagaatgtgaaaattccagtg

SEQ ID NO: 21 - DNA, 43 bp, Artificial Sequence
  Synthetic construct
tcagacgtgtgctcttccgatctgcatatttgccattttggat

SEQ ID NO: 22 - DNA, 43 bp, Artificial Sequence
  Synthetic construct
tcagacgtgtgctcttccgatcttgacaagattttcccttacc

SEQ ID NO: 23 - DNA, 39 bp, Artificial Sequence
  Synthetic construct
tcagacgtgtgctcttccgatctcacgcatacggtttgg

SEQ ID NO: 24 - DNA, 38 bp, Artificial Sequence
  Synthetic construct
tcagacgtgtgctcttccgatctcagtccggcttggag

SEQ ID NO: 25 - DNA, 38 bp, Artificial Sequence
  Synthetic construct
tcagacgtgtgctcttccgatctaggagctggtggagg

SEQ ID NO: 26 - DNA, 44 bp, Artificial Sequence
  Synthetic construct
tcagacgtgtgctcttccgatcttgacaacatagttggaatcac

SEQ ID NO: 27 - DNA, 41 bp, Artificial Sequence
  Synthetic construct
tcagacgtgtgctcttccgatctctgtgtcctttcaggatg

SEQ ID NO: 28 - DNA, 43 bp, Artificial Sequence
  Synthetic construct
tcagacgtgtgctcttccgatctagcagtgtatccattttctt

SEQ ID NO: 29 - DNA, 43 bp, Artificial Sequence
  Synthetic construct
tcagacgtgtgctcttccgatctgacctcaattttgtttcagg

SEQ ID NO: 30 - DNA, 40 bp, Artificial Sequence
  Synthetic construct
tcagacgtgtgctcttccgatcttaccaaccaagaagggg

SEQ ID NO: 31 - DNA, 38 bp, Artificial Sequence
  Synthetic construct
tcagacgtgtgctcttccgatctatggtcagcgcactc

SEQ ID NO: 32 - DNA, 43 bp, Artificial Sequence
  Synthetic construct
tcagacgtgtgctcttccgatctaacatgacttacttgatccc

SEQ ID NO: 33 - DNA, 47 bp, Artificial Sequence
  Synthetic construct
tcagacgtgtgctcttccgatctcacaagcatttggttttaaattat

SEQ ID NO: 34 - DNA, 40 bp, Artificial Sequence
  Synthetic construct
tcagacgtgtgctcttccgatctctcttacccactccagg

SEQ ID NO: 35 - DNA, 40 bp, Artificial Sequence
  Synthetic construct
tcagacgtgtgctcttccgatctagtcaggctggagaatc

SEQ ID NO: 36 - DNA, 41 bp, Artificial Sequence
  Synthetic construct
tcagacgtgtgctcttccgatctccttactcatggtcggat

SEQ ID NO: 37 - DNA, 41 bp, Artificial Sequence
  Synthetic construct
tcagacgtgtgctcttccgatctgtatcgtcaaggcactct

SEQ ID NO: 38 - DNA, 42 bp, Artificial Sequence
  Synthetic construct
tcagacgtgtgctcttccgatctgttgctgattttggtcttg

SEQ ID NO: 39 - DNA, 43 bp, Artificial Sequence
  Synthetic construct
tcagacgtgtgctcttccgatctacaatattcgctccatcttt

SEQ ID NO: 40 - DNA, 36 bp, Artificial Sequence
  Synthetic construct
tcagacgtgtgctcttccgatcttcagcgccgtcct

SEQ ID NO: 41 - DNA, 38 bp, Artificial Sequence
  Synthetic construct
tcagacgtgtgctcttccgatctcgagctggaccactg

SEQ ID NO: 42 - DNA, 43 bp, Artificial Sequence
  Synthetic construct
tcagacgtgtgctcttccgatctatgtctatgaagtgttgtgg

SEQ ID NO: 43 - DNA, 43 bp, Artificial Sequence
  Synthetic construct
tcagacgtgtgctcttccgatctcatgtattggtctctcatgg

SEQ ID NO: 44 - DNA, 43 bp, Artificial Sequence
  Synthetic construct
tcagacgtgtgctcttccgatcttgtgaagatctgtgactttg

SEQ ID NO: 45 - DNA, 44 bp, Artificial Sequence
  Synthetic construct
tcagacgtgtgctcttccgatctacaatcttttgatgacattgc

SEQ ID NO: 46 - DNA, 43 bp, Artificial Sequence
  Synthetic construct
tcagacgtgtgctcttccgatctatttaaccatgcagatcctc

SEQ ID NO: 47 - DNA, 43 bp, Artificial Sequence
  Synthetic construct
tcagacgtgtgctcttccgatctttcatgatgtttccttcgta

SEQ ID NO: 48 - DNA, 41 bp, Artificial Sequence
  Synthetic construct
tcagacgtgtgctcttccgatctccctaccttgtcaccaat

SEQ ID NO: 49 - DNA, 40 bp, Artificial Sequence
  Synthetic construct
tcagacgtgtgctcttccgatctcacccacagatccactg

SEQ ID NO: 50 - DNA, 43 bp, Artificial Sequence
  Synthetic construct
tcagacgtgtgctcttccgatcttactcaggatgagttttgtg

SEQ ID NO: 51 - DNA, 42 bp, Artificial Sequence
  Synthetic construct
tcagacgtgtgctcttccgatcttctgtacaagagatacccc

SEQ ID NO: 52 - DNA, 41 bp, Artificial Sequence
  Synthetic construct
tcagacgtgtgctcttccgatctatgtttggaactggcatc

SEQ ID NO: 53 - DNA, 36 bp, Artificial Sequence
  Synthetic construct
tcagacgtgtgctcttccgatctgcgcggacgagga

SEQ ID NO: 54 - DNA, 41 bp, Artificial Sequence
  Synthetic construct
tcagacgtgtgctcttccgatctcgcaaatttccttccact

SEQ ID NO: 55 - DNA, 41 bp, Artificial Sequence
  Synthetic construct
tcagacgtgtgctcttccgatctctttgcttgtcccgatag

SEQ ID NO: 56 - DNA, 44 bp, Artificial Sequence
  Synthetic construct
tcagacgtgtgctcttccgatcttggaaaaatagcctcaattct

SEQ ID NO: 57 - DNA, 42 bp, Artificial Sequence
  Synthetic construct
tcagacgtgtgctcttccgatctagtaattgaaccagtaggc

SEQ ID NO: 58 - DNA, 45 bp, Artificial Sequence
  Synthetic construct
tcagacgtgtgctcttccgatctaaggaaactgaattcaaaaaga

SEQ ID NO: 59 - DNA, 46 bp, Artificial Sequence
  Synthetic construct
acactctttccctacacgacgctcttccgatctcacggccaccgtc

SEQ ID NO: 60 - DNA, 52 bp, Artificial Sequence
  Synthetic construct
acactctttccctacacgacgctcttccgatctcaggctgtatttcttccac

SEQ ID NO: 61 - DNA, 47 bp, Artificial Sequence
  Synthetic construct
acactctttccctacacgacgctcttccgatcttctcaccacccgca

SEQ ID NO: 62 - DNA, 54 bp, Artificial Sequence
  Synthetic construct
acactctttccctacacgacgctcttccgatctagaagtacatctgctaaacat

SEQ ID NO: 63 - DNA, 55 bp, Artificial Sequence
  Synthetic construct
acactctttccctacacgacgctcttccgatctcagaaagaatgtctttgagtag

SEQ ID NO: 64 - DNA, 53 bp, Artificial Sequence
  Synthetic construct
acactctttccctacacgacgctcttccgatctcatgaagacctcacagtaaa

SEQ ID NO: 65 - DNA, 51 bp, Artificial Sequence
  Synthetic construct
acactctttccctacacgacgctcttccgatctgttatggaactgctcacc

SEQ ID NO: 66 - DNA, 46 bp, Artificial Sequence
  Synthetic construct
acactctttccctacacgacgctcttccgatctacgtgcgcgatgc

SEQ ID NO: 67 - DNA, 48 bp, Artificial Sequence
  Synthetic construct
acactctttccctacacgacgctcttccgatctggatatcgcccagcc

SEQ ID NO: 68 - DNA, 52 bp, Artificial Sequence
  Synthetic construct
acactctttccctacacgacgctcttccgatctttaccactcagagaaggag

SEQ ID NO: 69 - DNA, 54 bp, Artificial Sequence
  Synthetic construct
acactctttccctacacgacgctcttccgatctttctgcatggtattctttctc

SEQ ID NO: 70 - DNA, 49 bp, Artificial Sequence
  Synthetic construct
acactctttccctacacgacgctcttccgatctttgttggctttggggg

SEQ ID NO: 71 - DNA, 53 bp, Artificial Sequence
  Synthetic construct
acactctttccctacacgacgctcttccgatctaaagatggaaactttggact

SEQ ID NO: 72 - DNA, 54 bp, Artificial Sequence
  Synthetic construct
acactctttccctacacgacgctcttccgatctgtatacacaccttatatgggc

SEQ ID NO: 73 - DNA, 51 bp, Artificial Sequence
  Synthetic construct
acactctttccctacacgacgctcttccgatctcatagatgctctcccctc

SEQ ID NO: 74 - DNA, 52 bp, Artificial Sequence
  Synthetic construct
acactctttccctacacgacgctcttccgatctctcctttcttccctctctc

SEQ ID NO: 75 - DNA, 50 bp, Artificial Sequence
  Synthetic construct
acactctttccctacacgacgctcttccgatctgtagctgaggatgcctg

SEQ ID NO: 76 - DNA, 54 bp, Artificial Sequence
  Synthetic construct
acactctttccctacacgacgctcttccgatctaaagtggtgaagatatgtgac

SEQ ID NO: 77 - DNA, 49 bp, Artificial Sequence
  Synthetic construct
acactctttccctacacgacgctcttccgatcttggatccacttcctcc

SEQ ID NO: 78 - DNA, 52 bp, Artificial Sequence
  Synthetic construct
acactctttccctacacgacgctcttccgatctttaaccttgcagaatggtc

SEQ ID NO: 79 - DNA, 52 bp, Artificial Sequence
  Synthetic construct
acactctttccctacacgacgctcttccgatctacttggtctcaaagattcc

SEQ ID NO: 80 - DNA, 50 bp, Artificial Sequence
  Synthetic construct
acactctttccctacacgacgctcttccgatctcctggaacaggatctgc

SEQ ID NO: 81 - DNA, 53 bp, Artificial Sequence
  Synthetic construct
acactctttccctacacgacgctcttccgatctgatgacggaatataagctgg

SEQ ID NO: 82 - DNA, 52 bp, Artificial Sequence
  Synthetic construct
acactctttccctacacgacgctcttccgatctagtggatgggtaaaaccta

SEQ ID NO: 83 - DNA, 55 bp, Artificial Sequence
  Synthetic construct
acactctttccctacacgacgctcttccgatctaaagcctgtagttttacttact

SEQ ID NO: 84 - DNA, 50 bp, Artificial Sequence
  Synthetic construct
acactctttccctacacgacgctcttccgatctcagccccaatcccaata

SEQ ID NO: 85 - DNA, 53 bp, Artificial Sequence
  Synthetic construct
acactctttccctacacgacgctcttccgatctgcagaacttttaaagctgat

SEQ ID NO: 86 - DNA, 51 bp, Artificial Sequence
  Synthetic construct
acactctttccctacacgacgctcttccgatctgggtactcacgtttcctt

SEQ ID NO: 87 - DNA, 56 bp, Artificial Sequence
  Synthetic construct
acactctttccctacacgacgctcttccgatcttatttttattataaggcctgctg

SEQ ID NO: 88 - DNA, 50 bp, Artificial Sequence
  Synthetic construct
acactctttccctacacgacgctcttccgatctcagctttgcacctgttt

SEQ ID NO: 89 - DNA, 54 bp, Artificial Sequence
  Synthetic construct
acactctttccctacacgacgctcttccgatcttgatggaatgataaaccaaga

SEQ ID NO: 90 - DNA, 49 bp, Artificial Sequence
  Synthetic construct
acactctttccctacacgacgctcttccgatctgggcggtacctgtagt

SEQ ID NO: 91 - DNA, 49 bp, Artificial Sequence
  Synthetic construct
acactctttccctacacgacgctcttccgatcttacaggtgcctgagca

SEQ ID NO: 92 - DNA, 58 bp, Artificial Sequence
  Synthetic construct
acactctttccctacacgacgctcttccgatctgaaataagacggaaaattttttaac

SEQ ID NO: 93 - DNA, 53 bp, Artificial Sequence
  Synthetic construct
acactctttccctacacgacgctcttccgatctgtttgttggacatactggat

SEQ ID NO: 94 - DNA, 52 bp, Artificial Sequence
  Synthetic construct
acactctttccctacacgacgctcttccgatctgcctttcgacacatagttc

SEQ ID NO: 95 - DNA, 50 bp, Artificial Sequence
  Synthetic construct
acactctttccctacacgacgctcttccgatctaagcctcttgctcagtt

SEQ ID NO: 96 - DNA, 52 bp, Artificial Sequence
  Synthetic construct
acactctttccctacacgacgctcttccgatcttgagggaactcaaagtaca

SEQ ID NO: 97 - DNA, 53 bp, Artificial Sequence
  Synthetic construct
acactctttccctacacgacgctcttccgatctgataaatcggtactgtgctt

SEQ ID NO: 98 - DNA, 52 bp, Artificial Sequence
  Synthetic construct
acactctttccctacacgacgctcttccgatctaatccgtaagggtgaacta

SEQ ID NO: 99 - DNA, 49 bp, Artificial Sequence
  Synthetic construct
acactctttccctacacgacgctcttccgatctggagaagaggacagcg

SEQ ID NO: 100 - DNA, 50 bp, Artificial Sequence
  Synthetic construct
acactctttccctacacgacgctcttccgatcttcaatccagcaaggtgt

SEQ ID NO: 101 - DNA, 53 bp, Artificial Sequence
  Synthetic construct
acactctttccctacacgacgctcttccgatcttgcaactattttcttcctct

SEQ ID NO: 102 - DNA, 50 bp, Artificial Sequence
  Synthetic construct
acactctttccctacacgacgctcttccgatctgtacgcctccagatgag

SEQ ID NO: 103 - DNA, 53 bp, Artificial Sequence
  Synthetic construct
acactctttccctacacgacgctcttccgatctgaagtcctgagtgtagatga

SEQ ID NO: 104 - DNA, 52 bp, Artificial Sequence
  Synthetic construct
acactctttccctacacgacgctcttccgatctcctcactgattgctcttag

SEQ ID NO: 105 - DNA, 49 bp, Artificial Sequence
  Synthetic construct
acactctttccctacacgacgctcttccgatctagaagcccatcgtgtg

SEQ ID NO: 106 - DNA, 50 bp, Artificial Sequence
  Synthetic construct
acactctttccctacacgacgctcttccgatctgggtcccatcagtttga

SEQ ID NO: 107 - DNA, 57 bp, Artificial Sequence
  Synthetic construct
acactctttccctacacgacgctcttccgatctctttatggttatttgcattttaga

SEQ ID NO: 108 - DNA, 50 bp, Artificial Sequence
  Synthetic construct
acactctttccctacacgacgctcttccgatctaccttatacaccgtgcc

SEQ ID NO: 109 - DNA, 46 bp, Artificial Sequence
  Synthetic construct
caagcagaagacggcatacgagatnnnnnnngtctcgtgggctcgg

SEQ ID NO: 110 - DNA, 48 bp, Artificial Sequence
  Synthetic construct
aatgatacggcgaccaccgagatctacacnnnnntcgtcggcagcgtc

SEQ ID NO: 111 - DNA, 64 bp, Artificial Sequence
  Synthetic construct
caagcagaagacggcatacgagatgccaatgtgactggagttcagacgtgtgctcttccgatct

SEQ ID NO: 112 - DNA, 64 bp, Artificial Sequence
  Synthetic construct
caagcagaagacggcatacgagatcttgtagtgactggagttcagacgtgtgctcttccgatct

SEQ ID NO: 113 - DNA, 64 bp, Artificial Sequence
  Synthetic construct
caagcagaagacggcatacgagatcgtgatgtgactggagttcagacgtgtgctcttccgatct

SEQ ID NO: 114 - DNA, 64 bp, Artificial Sequence
  Synthetic construct
caagcagaagacggcatacgagatacatcggtgactggagttcagacgtgtgctcttccgatct

SEQ ID NO: 115 - DNA, 64 bp, Artificial Sequence
  Synthetic construct
caagcagaagacggcatacgagatgcctaagtgactggagttcagacgtgtgctcttccgatct

SEQ ID NO: 116 - DNA, 64 bp, Artificial Sequence
  Synthetic construct
caagcagaagacggcatacgagattggtcagtgactggagttcagacgtgtgctcttccgatct

SEQ ID NO: 117 - DNA, 64 bp, Artificial Sequence
  Synthetic construct
caagcagaagacggcatacgagatcactgtgtgactggagttcagacgtgtgctcttccgatct

SEQ ID NO: 118 - DNA, 64 bp, Artificial Sequence
  Synthetic construct
caagcagaagacggcatacgagatattggcgtgactggagttcagacgtgtgctcttccgatct

SEQ ID NO: 119 - DNA, 64 bp, Artificial Sequence
  Synthetic construct
caagcagaagacggcatacgagatgatctggtgactggagttcagacgtgtgctcttccgatct

SEQ ID NO: 120 - DNA, 64 bp, Artificial Sequence
  Synthetic construct
caagcagaagacggcatacgagattcaagtgtgactggagttcagacgtgtgctcttccgatct

SEQ ID NO: 121 - DNA, 64 bp, Artificial Sequence
  Synthetic construct
caagcagaagacggcatacgagatctgatcgtgactggagttcagacgtgtgctcttccgatct

SEQ ID NO: 122 - DNA, 64 bp, Artificial Sequence
  Synthetic construct
caagcagaagacggcatacgagataagctagtgactggagttcagacgtgtgctcttccgatct

SEQ ID NO: 123 - DNA, 64 bp, Artificial Sequence
  Synthetic construct
caagcagaagacggcatacgagatgtagccgtgactggagttcagacgtgtgctcttccgatct

SEQ ID NO: 124 - DNA, 64 bp, Artificial Sequence
  Synthetic construct
caagcagaagacggcatacgagattacaaggtgactggagttcagacgtgtgctcttccgatct

SEQ ID NO: 125 - DNA, 64 bp, Artificial Sequence
  Synthetic construct
caagcagaagacggcatacgagatttgactgtgactggagttcagacgtgtgctcttccgatct

SEQ ID NO: 126 - DNA, 64 bp, Artificial Sequence
  Synthetic construct
caagcagaagacggcatacgagatggaactgtgactggagttcagacgtgtgctcttccgatct

SEQ ID NO: 127 - DNA, 64 bp, Artificial Sequence
  Synthetic construct
caagcagaagacggcatacgagattgacatgtgactggagttcagacgtgtgctcttccgatct

SEQ ID NO: 128 - DNA, 64 bp, Artificial Sequence
  Synthetic construct
caagcagaagacggcatacgagatggacgggtgactggagttcagacgtgtgctcttccgatct

SEQ ID NO: 129 - DNA, 64 bp, Artificial Sequence
  Synthetic construct
caagcagaagacggcatacgagatgcggacgtgactggagttcagacgtgtgctcttccgatct

SEQ ID NO: 130 - DNA, 64 bp, Artificial Sequence
  Synthetic construct
caagcagaagacggcatacgagattttcacgtgactggagttcagacgtgtgctcttccgatct

SEQ ID NO: 131 - DNA, 64 bp, Artificial Sequence
  Synthetic construct
caagcagaagacggcatacgagatggccacgtgactggagttcagacgtgtgctcttccgatct

SEQ ID NO: 132 - DNA, 64 bp, Artificial Sequence
  Synthetic construct
caagcagaagacggcatacgagatcgaaacgtgactggagttcagacgtgtgctcttccgatct

SEQ ID NO: 133 - DNA, 64 bp, Artificial Sequence
  Synthetic construct
caagcagaagacggcatacgagatcgtacggtgactggagttcagacgtgtgctcttccgatct

SEQ ID NO: 134 - DNA, 64 bp, Artificial Sequence
  Synthetic construct
caagcagaagacggcatacgagatccactcgtgactggagttcagacgtgtgctcttccgatct

SEQ ID NO: 135 - DNA, 64 bp, Artificial Sequence
  Synthetic construct
caagcagaagacggcatacgagatatcagtgtgactggagttcagacgtgtgctcttccgatct

SEQ ID NO: 136 - DNA, 64 bp, Artificial Sequence
  Synthetic construct
caagcagaagacggcatacgagataggaatgtgactggagttcagacgtgtgctcttccgatct

SEQ ID NO: 137 - DNA, 33 bp, Artificial Sequence; identical to SEQ ID NO: 7
  Synthetic construct

SEQ ID NO: 138 - DNA, 33 bp, Artificial Sequence
  Synthetic contstruct
gatcggaagagcacacgtctgaactccagtcac

SEQ ID NO: 139 - DNA, 34 bp, Artificial Sequence
  Synthetic construct
gtgactggagttcagacgtgtgctcttccgatct

SEQ ID NO: 140 - DNA, 64 bp, Artificial Sequence; identical to SEQ ID NO: 6
  Synthetic construct

SEQ ID NO: 141 - DNA, 23 bp, Artificial Sequence
  Synthetic Construct
tcagacgtgtgctcttccgatct

SEQ ID NO: 142 - DNA, 33 bp, Artificial Sequence; identical to SEQ ID NO: 7
  Synthetic construct

SEQ ID NO: 143 - DNA, 58 bp, Artificial Sequence; identical to SEQ ID NO: 1
  Synthetic construct

SEQ ID NO: 144 - DNA, 64 bp, Artificial Sequence
  Synthetic construct
agatcggaagagcacacgtctgaactccagtcacnnnnnnatctcgtatgccgtcttctgcttg

SEQ ID NO: 145 - DNA, 58 bp, Artificial Sequence
  Synthetic construct
agatcggaagagcgtcgtgtagggaaagagtgtagatctcggtggtcgccgtatcatt

SEQ ID NO: 146 - DNA, 64 bp, Artificial Sequence; identical to SEQ ID NO: 6
  Synthetic construct

(19) United States
(12) Patent Application Publication
(10) Pub. No.: US 2017/0191127 A1
(43) Pub. Date: Jul. 6, 2017

(54) DROPLET PARTITIONED PCR-BASED LIBRARY PREPARATION

(71) Applicant: Bio-Rad Laboratories, Inc., Hercules, CA (US)

(72) Inventors: Shawn Hodges, Newark, CA (US); Nicholas Heredia, Mountain House, CA (US)

(21) Appl. No.: 15/394,396

(22) Filed: Dec. 29, 2016

Related U.S. Application Data
(60) Provisional application No. 62/272,874, filed on Dec. 30, 2015.

(51) Int. Cl. C12Q 1/68; C12N 15/10

(52) U.S. Cl. CPC C12Q 1/6874; C12N 15/1068

(57) ABSTRACT

Methods of preparing a target gene-enriched library are provided. In one aspect, the method comprises partitioning polynucleotide fragments into a plurality of partitions, wherein each partition further comprises a plurality of primer pairs for amplifying a target gene and wherein the primers comprise a portion of an adapter sequence; amplifying a target gene sequence to generate an amplicon comprising the target gene sequence flanked on either end by a portion of an adapter sequence; purifying the amplicon; and amplifying the amplicon using primers comprising full-length adapter sequences.

### CROSS-REFERENCE TO RELATED APPLICATIONS

This application claims priority to U.S. Provisional Application No. 62/272,874, filed Dec. 30, 2015, the entire content of which is incorporated by reference herein.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 094868-111210US-1032581_SequenceListing.txt, created on Dec. 28, 2016, 31,341 bytes, machine format IBM-PC, MS-Windows operating system, is hereby incorporated by reference in its entirety for all purposes.

### BACKGROUND OF THE INVENTION

Targeted sequencing allows for the investigation of selected genes, gene regions, or genomic elements in a genomic sample, enhancing the efficiency of next-generation sequencing. For enriching a target region before sequencing, several methods are used, including hybridization capture from sequencing libraries using target probes and the generation of sequencing libraries by PCR amplification of sample DNA using target specific primers. The generation of libraries by PCR amplification inherently introduces substantial amplification bias, which results in variable coverage of sequences and significantly affects quantification accuracy.

### BRIEF SUMMARY OF THE INVENTION

In one aspect, methods of preparing a target gene-enriched library are provided. In some embodiments, the method comprises:
(a) providing a plurality of polynucleotide fragments;
(b) partitioning the polynucleotide fragments into a plurality of partitions, wherein each partition further comprises a plurality of primer pairs, each primer pair comprising a forward primer and a reverse primer for amplifying a target gene, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence;
(c) amplifying a target gene sequence of a polynucleotide fragment in a partition with one of the primer pairs in the partition, thereby generating an amplicon comprising the target gene sequence flanked on the 5′ end by the portion of the first adapter sequence and flanked on the 3′ end by the portion of the second adapter sequence;
(d) purifying the amplicon; and
(e) amplifying the amplicon using a first amplicon primer comprising at least a portion of the first adapter sequence and a second amplicon primer comprising at least a portion of the second adapter sequence.

In some embodiments, the polynucleotide fragments are genomic DNA fragments. In some embodiments, the polynucleotide fragments are at least about 100 nucleotides in length. In some embodiments, the polynucleotide fragments are up to about 2000, up to about 5000, up to about 10,000, up to about 25,000, or up to about 50,000 nucleotides in length. In some embodiments, the polynucleotide fragments are about 100 to about 2000 nucleotides in length.

In some embodiments, in the partitioning step (b), each partition comprises at least 20 primer pairs. In some embodiments, each partition comprises at least 50 primer pairs. In some embodiments, each partition comprises at least 200 primer pairs. In some embodiments, each partition comprises at least 500 primer pairs.

In some embodiments, a target gene or gene region for amplification is a gene or gene region having a rare mutation. In some embodiments, a target gene or gene region for amplification is a gene or gene region that is associated with a cancer or an inherited disease.

In some embodiments, the first adapter sequence is a P7 adapter sequence and the second adapter sequence is a P5 adapter sequence. In some embodiments, the first adapter sequence is a P5 adapter sequence and the second adapter sequence is a P7 adapter sequence. In some embodiments, the P7 adapter sequence is a sequence having at least 70% identity (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identity) to SEQ ID NO:4. In some embodiments, the P7 adapter sequence is SEQ ID NO:4. In some embodiments, the P5 adapter sequence is a sequence having at least 70% identity (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identity) to SEQ ID NO:1. In some embodiments, the P5 adapter sequence is SEQ ID NO:1.

In some embodiments, for a forward primer or a reverse primer comprising a portion of the first adapter sequence, the portion of the first adapter sequence comprises at least 20 contiguous nucleotides of the first adapter sequence. In some embodiments, the portion of the first adapter sequence has at least 70% identity (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identity) to SEQ ID NO:7 or SEQ ID NO:8. In some embodiments, the portion of the first adapter sequence has the sequence of SEQ ID NO:7 or SEQ ID NO:8.

In some embodiments, the first adapter sequence and/or the second adapter sequence comprises a barcode sequence. In some embodiments, the first adapter sequence and/or the second adapter sequence comprising a barcode sequence has at least 70% identity (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identity) to SEQ ID NO:3 or SEQ ID NO:6.

In some embodiments, the forward primer for amplifying the target gene has at least 70% identity (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identity) to any of SEQ ID NOs:9-58 (e.g., SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, or SEQ ID NO:58). In some embodiments, the forward primer for amplifying the target gene comprises any of SEQ ID NOs:9-58.

In some embodiments, the reverse primer for amplifying the target gene has at least 70% identity (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identity) to any of SEQ ID NOs:59-108 (e.g., SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, or SEQ ID NO:108). In some embodiments, the reverse primer for amplifying the target gene comprises any of SEQ ID NOs:59-108.

In some embodiments, the first amplicon primer has at least 70% identity (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identity) to any of SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, or SEQ ID NO:136. In some embodiments, the first amplicon primer comprises any of SEQ ID NO:111-136. In some embodiments, the second amplicon primer has at least 70% identity (e.g., at least 70%, at least 75%, at least 80%, at least 85%, at least 90%, at least 91%, at least 92%, at least 93%, at least 94%, at least 95%, at least 96%, at least 97%, at least 98%, or at least 99% identity) to SEQ ID NO:1. In some embodiments, the second amplicon primer comprises SEQ ID NO:1.

In some embodiments, the partitions are droplets. In some embodiments, the partitions comprise an average volume of about 50 picoliters to about 2 nanoliters. In some embodiments, the partitions comprise an average volume of about 0.5 nanoliters to about 2 nanoliters. In some embodiments, the partitions comprise an average of about 0.1 to about 10 targets per droplet. In some embodiments, the partitions comprise an average of about 1 to about 5 targets per droplet.

In some embodiments, in the partitioning step (b), each partition further comprises one or more members selected from the group consisting of salts, nucleotides, buffers, stabilizers, DNA polymerase, detectable agents, and nuclease-free water. In some embodiments, the DNA polymerase is a high-fidelity DNA polymerase.

In some embodiments, the amplifying step (c) (also referred to herein as “target-specific” amplification) comprises from 1 to 30 cycles of amplification, e.g., from 5 to 30 cycles, from 10 to 30 cycles, from 15 to cycles, or from 10 to 25 cycles. In some embodiments, the amplifying step (c) comprises at least one cycle of amplification. In some embodiments, the amplifying step (c) comprises at least 5 cycles of amplification, at least 10 cycles of amplification, at least 15 cycles of amplification, at least 20 cycles of amplification, or at least 25 cycles of amplification. In some embodiments, the amplification step (c) comprises about 30 cycles of amplification.

In some embodiments, the amplifying step (e) (also referred to herein as “nested” amplification) comprises from 1 to 30 cycles of amplification, e.g., from 5 to 30 cycles, from 10 to 30 cycles, from 15 to cycles, or from 10 to 25 cycles. In some embodiments, the amplifying step (e) comprises at least one cycle of amplification, at least 5 cycles of amplification, at least 10 cycles of amplification, at least 15 cycles of amplification, at least 20 cycles of amplification, or at least 25 cycles of amplification. In some embodiments, the amplification step (e) comprises about 30 cycles of amplification.

In some embodiments, following the amplifying step (e), the method further comprises purifying the amplicons. In some embodiments, the purifying step comprises breaking the partitions and separating the amplicon from at least one other component in the partition. In some embodiments, following the amplifying step (e), the method further comprises sequencing at least one amplicon.

In another aspect, libraries of amplicons generated according to a method as described herein are provided.

In another aspect, kits for preparing a target gene-enriched library are provided. In some embodiments, the kit comprises:
(a) a first composition for partitioning into a plurality of partitions, wherein the composition comprises a plurality of primer pairs, each primer pair comprising a forward primer and a reverse primer for amplifying a target gene, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence; and
(b) a second composition comprising a first primer and a second primer, wherein the first primer comprises the first adapter sequence and the second primer comprises the second adapter sequence.

In another aspect, methods for detecting a plurality of targets in a biological sample are provided. In some embodiments, the method comprises:
(a) obtaining a plurality of polynucleotide fragments from the biological sample;
(b) partitioning the polynucleotide fragments into a plurality of partitions, wherein each partition further comprises a plurality of primer pairs, each primer pair comprising a forward primer and a reverse primer for amplifying a target gene, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence;
(c) amplifying a target gene sequence of a polynucleotide fragment in a partition with one of the primer pairs in the partition, thereby generating an amplicon comprising the target gene sequence flanked on the 5′ end by the portion of the first adapter sequence and flanked on the 3′ end by the portion of the second adapter sequence;
(d) purifying the amplicon;
(e) amplifying the amplicon using a first primer comprising the first adapter sequence and a second primer comprising the second adapter sequence; and
(f) detecting a plurality of amplicons from the amplifying step (e).

In some embodiments, the detecting step comprises sequencing the plurality of amplicons. In some embodiments, the sequencing is sequencing by synthesis.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Spring Harbor Lab Press (Cold Spring Harbor, N.Y. 1989). The term “a” or “an” is intended to mean “one or more.” The term “comprise,” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

As used herein, the term “adapter” is a polynucleotide sequence that is not native to target sequence (e.g., a target gene sequence), but that is added to the target sequence, such as in an amplification reaction. In some embodiments, an adapter comprises a hybridization sequence that can hybridize to a complementary or substantially complementary capture probe, such as a capture probe immobilized to a solid surface. In some embodiments, an adapter comprises a sequence that can hybridize to a primer, such as a sequencing primer or an amplification primer.

The terms “partial” and “portion,” as used with reference to a sequence, refer to a length of the sequence that is less than the full length of the sequence. In some embodiments, a portion of a sequence can be from about 20% to about 80% of the full length of the sequence, about 25% to about 75% of the full length of the sequence, or about 30% to about 70% of the full length of the sequence, e.g., about 20%, about 30%, about 40%, about 50%, about 60%, about 70%, or about 80% of the full length of the sequence. In some embodiments, a portion of a sequence is a contiguous number of nucleotides of the sequence (e.g., at least 10, at least 15, at least 20, at least 25, at least 30, at least 40, or at least 50 or more contiguous nucleotides of the sequence). As a non-limiting example, in some embodiments, a polynucleotide comprising a portion of an adapter sequence comprises about 20% to about 80% of the full adapter sequence.

As used herein, the term “partitioning” or “partitioned” refers to separating a sample into a plurality of portions, or “partitions.” Partitions can be solid or fluid. In some embodiments, a partition is a solid partition, e.g., a microchannel. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

As used herein, a “target” refers to a polynucleotide sequence to be detected. In some embodiments, the target is a “target gene sequence,” which as used herein, refers to a gene or a portion of a gene to be detected. In some embodiments, a target is a polynucleotide sequence (e.g., a gene or a portion of a gene) having a mutation that is associated with a disease such as a cancer. In some embodiments, the target is a polynucleotide sequence having a rare mutation that is associated with a disease such as a cancer.

The term “nucleic acid amplification” or “amplification” refers to any in vitro method for multiplying the copies of a target sequence of nucleic acid in a linear or exponential manner. Such methods include, but are not limited to, polymerase chain reaction (PCR); DNA ligase chain reaction (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3SR); single-primer isothermal amplification (SPIA), loop mediated isothermal amplification (LAMP), strand displacement amplification (SDA); multiple displacement amplification (MDA); rolling circle amplification (RCA); as well as others known to those of skill in the art. See, e.g., Fakruddin et al., J. Pharm Bioallied Sci. 2013 5(4):245-252.

“Amplifying” refers to a step of submitting a solution (e.g., in droplets or in bulk) to conditions sufficient to allow for amplification of a polynucleotide to yield an amplification product or “amplicon.” Components of an amplification reaction include, e.g., primers, a polynucleotide template, polymerase, nucleotides, and the like. The term amplifying typically refers to an exponential increase in target nucleic acid. However, as used herein, the term amplifying can also refer to linear increases in the numbers of a particular target sequence of nucleic acid, such as is obtained with cycle sequencing.

The term “primer” refers to a polynucleotide sequence that hybridizes to a sequence on a target nucleic acid and serves as a point of initiation of nucleic acid synthesis. Primers can be of a variety of lengths. In some embodiments, a primer is less than 100 nucleotides in length, e.g., from about 10 to about 50, from about 15 to about 40, from about 15 to about 30, from about 20 to about 80, or from about 20 to about 60 nucleotides in length. The length and sequences of primers for use in an amplification reaction (e.g., PCR) can be designed based on principles known to those of skill in the art; see, e.g., PCR Protocols: A Guide to Methods and Applications, Innis et al., eds, 1990. In some embodiments, a primer comprises one or more modified or non-natural nucleotide bases. In some embodiments, a primer comprises a label (e.g., a detectable label).

A nucleic acid, or portion thereof, “hybridizes” to another nucleic acid under conditions such that non-specific hybridization is minimal at a defined temperature in a physiological buffer. In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, 18, 20, 22, 24, 26, 28, or 30 contiguous complementary nucleotides, including “universal” nucleotides that are complementary to more than one nucleotide partner. Alternatively, a primer, or portion thereof, can hybridize to a primer binding site if there are fewer than 1 or 2 complementarity mismatches over at least about 12, 14, 16, 18, 20, 25, or 30 contiguous complementary nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 60, 65, 70, 75, or 80° C., e.g., about 45° C. to about 60° C., e.g., about 55° C.-59° C. In some embodiments, the defined temperature at which specific hybridization occurs is about 5° C. below the calculated melting temperature of the primers

As used herein, “nucleic acid” refers to DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

### BRIEF DESCRIPTION OF THE DRAWINGS

FIG. 1. An exemplary schematic depicting construction of target-enriched library. Genomic DNA fragments comprising a target gene of interest are partitioned into droplets. The droplets also contain forward and reverse primer pairs for amplifying target genes, in which the forward primer includes a partial P7 adapter sequence and the reverse primer includes a partial P5 adapter sequence. Droplet digital PCR (ddPCR) amplification is performed to yield droplets having an amplified target gene with partial P7 and partial P5 adapter sequences attached at the 5′ and 3′ ends, respectively, of the target gene. The droplets comprising the ddPCR amplicons are broken and the PCR amplicons are purified. The amplicons are then subjected to a nested PCR amplification reaction using a forward primer having a full-length P7 adapter sequence and a reverse primer having a full-length P5 adapter sequence. An “index” or barcode sequence can be included within the full-length adapter sequences. The resulting amplification product is a double-stranded polynucleotide comprising the target gene, a full-length P5 adapter, and a full-length P7 adapter.

FIG. 2. (SEQ ID NOs: 1, 142, 141, 140, 143-146, 7, 138, and 139) Schematic depicting an exemplary library preparation scheme using P5 and P7 adapters. For the first amplification step, a partial P7 target-specific forward primer (3′-Rev-GSP-TCTAGCCTTCTCGTGTGCAGACT-5′ SEQ ID NO: 141) and a partial P5 target-specific reverse primer (5′-ACACTCTTTCCCTACACGACGCTCTTCCGATCT-For-GSP-3′ SEQ ID NO: 142) are used to enrich for target genes. For the second amplification step, primers comprising a full-length barcoded P7 adapter sequence (“P7-Index-RD2”; 3′-TCTAGCCTTCTCGTGTGCAGACTTGAGGTCAGTG TAGAGCATACGGCAGA AGACGAAC-5′ SEQ ID NO: 140) and a full-length P5 adapter sequence (“P5-RD1”; 5′-AATGATACGGCGACCACCGAGATCTACACTCTTTCCCTACACGACGCTCTTCCGATC T-3′ SEQ ID NO: 1) are used. The sequences in green (for P5-RD1) and orange (for P7-Index-RD2) represent sequences that are complementary to capture oligonucleotides used for downstream sequencing steps. The sequences in purple and blue represent sequencing primer regions in the P5 and P7 adapter sequences, respectively. Exemplary sequencing primers include Multiplexing Read 1 Sequencing Primer (5′-ACACTCTTTCCCTACACGACGCTCTTCCGATCT-3′ SEQ ID NO: 137), Multiplexing Index Read Sequencing Primer (5′-GATCGGAAGAGCACACGTCTGAACTCCAGTCAC-3′ SEQ ID NO: 138), and Multiplexing Read 2 Sequencing Primer (3′-TCTAGCCTTCTCGTGTGCAGACTTGAGGTCAGTG-5′ SEQ ID NO: 139).

FIG. 3. Sequencing results of droplet partitioned vs. bulk amplification demonstrating improved uniformity of number of reads per target using droplet partitioning amplification.

FIG. 4A-B. (A) Experion Gel analysis of libraries prepared from recovered product from droplets in 200plex experiments. L=ladder in bp; D=material recovered from droplets; B=material recovered from bulk reactions. (B) Plot of the sizes of Adapted-Amplicons in the 200plex rank ordered from lowest to highest in bp.

FIG. 5A-B. (A) Size distribution of genomic DNA fragments used for target-specific PCR. (B) Size distribution of AMPure-purified DNA fragments post-nested PCR, derived from 15 cycles (“15TS”) or 30 cycles (“30TS”) of target-specific PCR in bulk vs. droplets.

FIG. 6. Upper panels: Sequencing metrics for sequencing reads obtained from target-specific PCR performed with Pre-Amp Supermix (left) vs. ddPCR Supermix (right). Bottom panel: Sequencing read counts for specified cancer targets obtained from target-specific PCR performed with Pre-Amp master mix (red) vs. ddPCR Supermix (blue).

FIG. 7. Normalized value by normalized stock library concentration (blue) or normalized sequencing read count (red) obtained from target-specific PCR performed with Pre-Amp Supermix or ddPCR Supermix for specific cancer targets.

FIG. 8. Read counts vs. library and cancer target. The y-axis reports a ration of the sequencing read counts for a 48-plex derived from libraries 8 vs. 9, in which the target-specific PCR step was performed in droplets vs. bulk, respectively (with ddPCR Supermix for probes, no dUTP) vs. the cancer targets on the x-axis.

### DETAILED DESCRIPTION OF THE INVENTION

### I. INTRODUCTION

Described herein are methods, compositions, and kits for preparing a target-enriched library from a sample. Polynucleotide fragments obtained from the sample are partitioned into a plurality of partitions and amplified in a first amplification reaction using primers that comprise partial adapter sequences. The amplification products of the first amplification reaction are recovered and are used as the template for a second amplification reaction using primers that comprise full-length adapter sequences. The methods described herein reduce the amplification bias that is inherently introduced by high-order multiplexing in PCR and provides a more uniform representation of amplicons from a sample for downstream detection (e.g., sequencing) applications.

### II. METHODS OF PREPARING TARGET-ENRICHED LIBRARIES

In one aspect, methods of preparing a target-enriched library are provided. In some embodiments, the method comprises:
(a) providing a plurality of polynucleotide fragments;
(b) partitioning the polynucleotide fragments into a plurality of partitions, wherein each partition further comprises a plurality of primer pairs, each primer pair comprising a forward primer and a reverse primer for amplifying a target gene, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence;
(c) amplifying a target gene sequence of a polynucleotide fragment in a partition with one of the primer pairs in the partition, thereby generating an amplicon comprising the target gene sequence flanked on the 5′ end by the portion of the first adapter sequence and flanked on the 3′ end by the portion of the second adapter sequence;
(d) purifying the amplicon; and
(e) amplifying the amplicon using a first primer comprising the first adapter sequence and a second primer comprising the second adapter sequence.

### Polynucleotide Fragments

The methods described herein can be used to generate libraries from any polynucleotide sequences of interest. The polynucleotides may be single stranded or double stranded, or may contain portions of both double stranded and single stranded sequences. For example, the polynucleotide sequences may be genomic DNA, cDNA, mRNA, or a combination or hybrid of DNA and RNA.

In some embodiments, the polynucleotide sequence (e.g., genomic DNA) is obtained from a sample such as a biological sample. Biological samples can be obtained from any biological organism, e.g., an animal, plant, fungus, pathogen (e.g., bacteria or virus), or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, and transformed cells, stem cells, stool, urine, etc.

In some embodiments, the polynucleotide sequences for generating target-enriched libraries are genomic DNA. In some embodiments, the polynucleotide sequences comprise a subset of a genome (e.g., selected genes that may harbor mutations for a particular population, such as individuals who are predisposed for a particular type of cancer). In some embodiments, the polynucleotide sequences comprise exome DNA, i.e., a subset of whole genomic DNA enriched for transcribed sequences which contains the set of exons in a genome. In some embodiments, the polynucleotide sequences comprise transcriptome DNA, i.e., the set of all mRNA or “transcripts” produced in a cell or population of cells.

In some embodiments, the polynucleotides are fragmented to produce polynucleotide fragments of one or more specific sizes. Any method of fragmentation can be used. In some embodiments, the polynucleotides are fragmented by mechanical means (e.g., ultrasonic cleavage, acoustic shearing, needle shearing, or sonication). In some embodiments, the polynucleotides are fragmented by chemical methods or by enzymatic methods (e.g., using endonucleases, such as dsDNA Fragmentase®, New England Biolabs, Inc., Ipswich, Mass.). In some embodiments, fragmentation is accomplished by ultrasound (e.g., Covaris or Sonicman 96-well format instruments). Methods of fragmentation are known in the art; see, e.g., US 2012/0004126.

In some embodiments, the polynucleotide fragments are subjected to a size selection step to obtain polynucleotide fragments having a certain size or range of sizes. Any methods of size selection can be used. For example, in some embodiments, fragmented polynucleotides are separated by gel electrophoresis and the band corresponding to a fragment size or range of sizes of interest is extracted from the gel. In some embodiments, a spin column can be used to select for fragments having a certain minimum size. In some embodiments, paramagnetic beads can be used to selectively bind DNA fragments having a desired range of sizes. In some embodiments, a combination of size selection methods can be used.

In some embodiments, polynucleotide fragments are selected that are at least about 100 nucleotides in length. In some embodiments, the polynucleotide fragments are up to about 1000 nucleotides in length, up to about 5000 nucleotides in length, up to about 10,000 nucleotides in length, up to about 20,000 nucleotides in length, up to about 30,000 nucleotides in length, up to about 40,000 nucleotides in length, or up to about 50,000 nucleotides in length.

In some embodiments, the polynucleotide fragments that are selected are from about 100 to about 50,000 nucleotides in length, e.g., from about 1000 to about 50,000, from about 5000 to about 50,000, from about 1000 to about 25,000, from about 5000 to about 25,000, from about 100 to about 10,000, from about 1000 to about 10,000, from about 100 to about 5000, from about 100 to about 2000, from about 100 to about 1500, from about 100 to about 1000, from about 100 to about 900, or from about 200 to about 800 nucleotides in length. In some embodiments, the polynucleotide fragmented polynucleotides (e.g., genomic DNA fragments) have an average length of about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, or about 2000 nucleotides.

### Adapters

The methods described herein are used to add adapters to the 5′ and 3′ ends of PCR amplicons from target genes or gene regions. Typically, adapters are synthetic nucleic acid sequences that are added to a target nucleotide sequence (e.g., a target gene or gene region). An adapter can vary in the length of the sequence. In some embodiments, an adapter has a length of about 20 nucleotides to about 500 nucleotides, e.g., from about 30 to about 350 nucleotides, from about 40 to about 200 nucleotides, from about 30 to about 150 nucleotides, from about 20 to about 200 nucleotides, or from about 20 to about 100 nucleotides (e.g., about 20, 30, 40, 50, 60, 70, 80, 90, 100, 120, 140, 160, 180, 200, 220, 240, 260, 280, 300, 320, 340, 360, 380, 400, 420, 440, 460, 480, or 500 nucleotides).

In some embodiments, an adapter sequence comprises a universal sequence. As used herein, a “universal” sequence refers to a region of nucleotide sequence that is common to a plurality of adapters (e.g., a region of nucleotide sequence that is common to a plurality of 5′ end adapters or a region of nucleotide sequence that is common to a plurality of 3′ end adapters). In some embodiments, the adapters comprise a variable sequence. For example, one 5′ end adapter can comprise a region of nucleotide sequence that differs from the corresponding region of another 5′ end adapter at one or more nucleotides, and one 3′ end adapter can comprise a region of nucleotide sequence that differs from the corresponding region of another 3′ end adapter at one or more nucleotides. In some embodiments, adapters can comprise a universal sequence region and a variable sequence region.

In some embodiments, adapters can comprise an “index” or “barcode” sequence. As used herein, an index or barcode sequence is a short nucleotide sequence (e.g., at least about 4, 6, 8, 10, or 12, nucleotides long) that identifies a molecule to which it is conjugated. In some embodiments, a barcode sequence is from about 4 nucleotides to about 20 nucleotides in length, about 6 nucleotides to about 12 nucleotides in length, or about 4 to about 10 nucleotides in length. The length of the barcode sequence determines how many unique samples can be differentiated. For example, a 1 nucleotide barcode can differentiate 4, or fewer, different samples or molecules; a 4 nucleotide barcode can differentiate 4⁴ or 256 samples or fewer; a 6 nucleotide barcode can differentiate 4096 different samples or fewer; and an 8 nucleotide barcode can index 65,536 different samples or fewer. In some embodiments, a barcode is used to identify molecules in a partition (a “partition-specific barcode”). A partition-specific barcode should be unique for that partition as compared to barcodes present in other partitions. In some embodiments, a barcode is used to identify a source of a nucleic acid (e.g., a cell or sample from which the nucleic acid is obtained). In some embodiments, a barcode is used to identify a molecule (e.g., target nucleic acid sequence) to which it is conjugated. In some embodiments, a barcode is used to discriminate samples when multiple samples are processed in parallel (e.g., for screening multiple patient samples by a cancer panel as described herein in which the samples are loaded simultaneously on a sequencer). Such an approach has the advantage of reducing the cost of sequencing by economies of scale. The use of barcode technology is well known in the art, see for example Katsuyuki Shiroguchi, et al. Proc Natl Acad Sci USA., 2012 Jan. 24; 109(4):1347-52; and Smith, A M et al., Nucleic Acids Research Can 11, (2010). Methods of designing and attaching barcode sequences for identifying a molecule (e.g., attaching a barcode to a polynucleotide sequence) are also described, for example, in U.S. Pat. No. 6,235,475, the entire content of which is incorporated by reference.

P5 and P7 Adapters

In some embodiments, a first adapter sequence is added to the 5′ end of the target gene or gene region, and a second adapter sequence is added to the 3′ end of the target gene or gene region. In some embodiments, the adapter sequences that are added to the 5′ and 3′ ends of target genes or gene regions are P5 adapter and P7 adapter sequences. The P5 and P7 adapters, which are utilized in Illumina sequencing chemistry (also known in the art as “bridge amplification”), are adapters that bind to complementary oligonucleotides on the surface of an array (e.g., a flowcell surface), thereby allowing library fragments bound to the P5 or P7 adapter to attach to the array surface. P5 and P7 adapter sequences are known in the art and are described, for example, in Bentley et al., Nature 456:53-59 (2008). See also, U.S. Pat. No. 8,192,930.

In some embodiments, a P5 adapter is added to the 5′ end of the target gene or gene region, and a P7 adapter is added to the 3′ end of the target gene or gene region. In some embodiments, a P7 adapter is added to the 5′ end of the target gene or gene region, and a P5 adapter is added to the 3′ end of the target gene or gene region.

In some embodiments, the P5 adapter sequence has the following sequence:

(SEQ ID NO: 1)
5′-AATGATACGGCGACCACCGAGATCTACACTCTTTCCCTACACG
ACGCTCTTCCGATCT-3′

In some embodiments, a P5 adapter sequence has at least 70% identity (e.g., at least 70%, 75%, 80%, 85%, 90%, 91%, 92%, 93%, 94%, 95%, 96%, 97%, 98%, or 99% identity) to SEQ ID NO:1. In some embodiments, a P5 adapter sequence having at least 70% identity to SEQ ID NO:1 comprises the contiguous nucleic acid sequence 5′-AATGATACGGCGACCACCGAGATCT (SEQ ID NO:2) from the P5 adapter sequence. In some embodiments, SEQ ID NO:2 is an invariant sequence at the 5′ end of the full-length P5 adapter that hybridizes to a capture oligonucleotide on a solid-phase surface (e.g., flow-cell) in a sequencing reaction.

In some embodiments, the P5 adapter sequence comprises an index or barcode sequence. In some embodiments, the index or barcode sequence comprises 4-20 nucleotides (e.g., 6-15, 6-12, 4-10, or about 4, 5, 6, 7, 8, 9, 10, 11, 12, 13, 14, 15, 16, 17, 18, 19, or 20 nucleotides). In some embodiments, a barcode sequence can be inserted within the sequence of SEQ ID NO:1. In some embodiments, a P5 adapter sequence comprising a barcode has the following sequence:

(SEQ ID NO: 3)
5′-AAT GAT ACG GCG ACC ACC GAG ATC TNN NNN NAC
ACT CTT TCC CTA CAC GAC GCT CTT CCG ATC T-3′

In some embodiments, a P5 adapter sequence comprising a barcode has at least 70% identity (e.g., at least 70%, 75%, 80%, 85%, 90%, 91%, 92%, 93%, 94%, 95%, 96%, 97%, 98%, or 99% identity) to SEQ ID NO:3.

In some embodiments, the P7 adapter sequence has the following sequence:

(SEQ ID NO: 4)
5-CAA GCA GAA GAC GGC ATA CGA GAT GTG ACT GGA
GTT CAG ACG TGT GCT CTT CCG ATC T-3′

In some embodiments, a P7 adapter sequence has at least 70% identity (e.g., at least 70%, 75%, 80%, 85%, 90%, 91%, 92%, 93%, 94%, 95%, 96%, 97%, 98%, or 99% identity) to SEQ ID NO:4. In some embodiments, a P7 adapter sequence having at least 70% identity to SEQ ID NO:4 comprises the contiguous nucleic acid sequence CAAGCAGAAGACGGCATACGAGAT (SEQ ID NO:5) from the P7 adapter sequence. In some embodiments, SEQ ID NO:5 is an invariant sequence at the 5′ end of the full-length P7 adapter that hybridizes to a capture oligonucleotide on a solid-phase surface (e.g., flow-cell) in a sequencing reaction.

In some embodiments, the P7 adapter sequence comprises an index or barcode sequence. In some embodiments, the index or barcode sequence comprises 4-20 nucleotides (e.g., 6-15, 6-12, 4-10, or about 4, 5, 6, 7, 8, 9, 10, 11, 12, 13, 14, 15, 16, 17, 18, 19, or 20 nucleotides). In some embodiments, a barcode sequence can be inserted within the sequence of SEQ ID NO:4. In some embodiments, a P7 adapter sequence comprising a barcode has the following sequence:

(SEQ ID NO: 6)
5-CAA GCA GAA GAC GGC ATA CGA GAT NNN NNN GTG
ACT GGA GTT CAG ACG TGT GCT CTT CCG ATC T-3′

In some embodiments, a P7 adapter sequence comprising a barcode has at least 70% identity (e.g., at least 70%, 75%, 80%, 85%, 90%, 91%, 92%, 93%, 94%, 95%, 96%, 97%, 98%, or 99% identity) to SEQ ID NO:6.

Other Adapter Sequences

In some embodiments, the adapter sequences that are added to the 5′ and 3′ ends of target genes or gene regions are Nextera adapters (Illumina). Nextera adapters are known in the art and are described, for example, in Turner, Front Genet., 2014, 5:5 (doi: 10.3389/fgene.2014.00005). In some embodiments, the adapter sequence is an “Index 1 Read” or an “Index 2 Read” sequence. In some embodiments, the Index 1 Read adapter sequence has the following sequence:

(SEQ ID NO: 109)
5′-CAAGCAGAAGACGGCATACGAGAT[i7]GTCTCGTGGGCTCG
G-3′

In some embodiments, an Index 1 Read adapter sequence has at least 70% identity (e.g., at least 70%, 75%, 80%, 85%, 90%, 91%, 92%, 93%, 94%, 95%, 96%, 97%, 98%, or 99% identity) to SEQ ID NO:109.

In some embodiments, the Index 2 Read adapter sequence has the following sequence:

(SEQ ID NO: 110)
5′-AATGATACGGCGACCACCGAGATCTACAC[i5]TCGTCGGCAG
CGTC-3′

In some embodiments, an Index 2 Read adapter sequence has at least 70% identity (e.g., at least 70%, 75%, 80%, 85%, 90%, 91%, 92%, 93%, 94%, 95%, 96%, 97%, 98%, or 99% identity) to SEQ ID NO:110.

In some embodiments, the adapter sequences that are added to the 5′ and 3′ ends of target genes or gene regions are adapter sequences that are commercially available, e.g., from Pacific Biosciences, Roche, or Ion Torrent. Adapters and adapter sequences are also described, for example, in US 2012/0196279, WO 2013/169998, and WO 2015/121236, incorporated by reference herein.

Partial Adapter Sequences

As further described below in the section “Reagents for Target-Specific Amplification Reaction,” a target-specific amplification reaction is performed using target-specific primer pairs for amplifying a target gene. In some embodiments, a target-specific primer pair comprises a forward primer and a reverse primer, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence. As used herein, a “partial” adapter sequence or a “portion” of an adapter sequence refers to a length of an adapter sequence that is less than the full length of the adapter sequence (e.g., a length of a P5 or P7 adapter sequence as described herein that is less than the full length of the P5 or P7 adapter sequence). In some embodiments, a portion of an adapter sequence can be from about 20% to about 80% of the full length of the adapter sequence, about 25% to about 75% of the full length of the adapter sequence, or about 30% to about 70% of the full length of the adapter sequence, e.g., about 20%, about 30%, about 40%, about 50%, about 60%, about 70%, or about 80% of the full length of the adapter sequence. In some embodiments, a “partial” or “portion” of an adapter sequence is a contiguous number of nucleotides of the adapter sequence (e.g., at least 10, at least 15, at least 20, at least 25, at least 30, at least 40, or at least 50 or more contiguous nucleotides of the adapter sequence, e.g., a P5 or P7 sequence as described herein).

In some embodiments, a partial P5 target-specific primer comprises at least 10, at least 15, at least 20, at least 25, at least 30, or at least 35 nucleotides of a P5 adapter of SEQ ID NO:1 or SEQ ID NO:3. In some embodiments, the partial P5 target-specific primer that comprises at least 10, at least 15, at least 20, at least 25, at least 30, or at least 35 nucleotides of a P5 adapter of SEQ ID NO:1 or SEQ ID NO:3 is a target-specific forward primer. In some embodiments, the partial P5 target-specific primer that comprises at least 10, at least 15, at least 20, at least 25, at least 30, or at least 35 nucleotides of a P5 adapter of SEQ ID NO:1 or SEQ ID NO:3 is a target-specific reverse primer. In some embodiments, a partial P5 target-specific primer comprises at least 10, at least 15, at least 20, at least 25, at least 30, or at least 35 nucleotides at the 3′ end of the P5 adapter of SEQ ID NO:1 or SEQ ID NO:3. In some embodiments, a partial P5 target-specific primer comprises a sequence having at least 70% identity (e.g., at least 70%, 75%, 80%, 85%, 90%, 91%, 92%, 93%, 94%, 95%, 96%, 97%, 98%, or 99% identity) to the sequence 5′-ACACTCTTTCCCTACACGACGCTCTTCCGATCT-3′ (SEQ ID NO:7). In some embodiments, a partial P5 target-specific primer comprises the sequence of SEQ ID NO:7.

In some embodiments, a partial P7 target-specific primer comprises at least 10, at least 15, at least 20, at least 25, at least 30, or at least 35 nucleotides of a P7 adapter of SEQ ID NO:4 or SEQ ID NO:6. In some embodiments, the partial P7 target-specific primer that comprises at least 10, at least 15, at least 20, at least 25, at least 30, or at least 35 nucleotides of a P7 adapter of SEQ ID NO:4 or SEQ ID NO:6 is a target-specific forward primer. In some embodiments, the partial P7 target-specific primer that comprises at least 10, at least 15, at least 20, at least 25, at least 30, or at least 35 nucleotides of a P7 adapter of SEQ ID NO:4 or SEQ ID NO:6 is a target-specific reverse primer. In some embodiments, a partial P7 target-specific primer comprises at least 10, at least 15, at least 20, at least 25, at least 30, or at least 35 nucleotides at the 3′ end of the P7 adapter of SEQ ID NO:4 or SEQ ID NO:6. In some embodiments, a partial P7 target-specific primer comprises a sequence having at least 70% identity (e.g., at least 70%, 75%, 80%, 85%, 90%, 91%, 92%, 93%, 94%, 95%, 96%, 97%, 98%, or 99% identity) to the sequence 5′-TCAGACGTGTGCTCTTCCGATCT-3′ (SEQ ID NO:8). In some embodiments, a partial P7 target-specific primer comprises the sequence of SEQ ID NO:8.

In some embodiments, a partial adapter sequence comprises at least 10, at least 15, at least 20, at least 25, at least 30 or more contiguous nucleotides of an Index 1 Read adapter sequence (SEQ ID NO:109) or Index 2 Read adapter sequence (SEQ ID NO:110) as described herein. In some embodiments, a partial Index 1 Read or Index 2 Read adapter sequence is a contiguous region at the 3′ end of the Index 1 Read or Index 2 Read sequence.

### Reagents for Target-Specific Amplification Reaction

For generating target-enriched libraries from polynucleotide fragments as described herein, a first amplification reaction is performed using primers that are specific for target genes or gene regions. In some embodiments, an amplification reaction comprises a plurality of primer pairs for enriching a plurality of target genes or gene regions.

Target-Specific Amplification Primers

In some embodiments, a primer pair for amplifying a target gene or gene region comprises a forward primer and a reverse primer, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence.

In some embodiments, the target genes or gene regions to be enriched for have known associations with a disease (e.g., a cancer, a neuromuscular disease, a cardiovascular disease, a developmental disease, or a metabolic disease),In some embodiments, the target genes or gene regions to be enriched for have known associations with a cancer, including but not limited to bladder cancer, brain cancer, breast cancer, cervical cancer, colorectal cancer, endometrial cancer, esophageal cancer, gastric cancer, head and neck cancer, kidney cancer, leukemia, liver cancer, lung cancer, lymphoma, melanoma, ovarian cancer, pancreatic cancer, prostate cancer, or thyroid cancer. Thus, in some embodiments, a target-specific amplification primer comprises a sequence that hybridizes to a target gene or gene region that has a known association with a cancer.

In some embodiments, the target genes or gene regions that are enriched for have known associations with a disease (e.g., an inherited disease), including but not limited to autism spectrum disorders, cardiomyopathy, ciliopathies, congenital disorders of glyosylation, congenital myasthenic syndromes, epilepsy and seizure disorders, eye disorders, glycogen storage disorders, hereditary cancer syndrome, hereditary periodic fever syndromes, inflammatory bowel disease, lysosomal storage disorders, multiple epiphyseal dysplasia, neuromuscular disorders, Noonan Syndrome and related disorders, perioxisome biogenesis disorders, or skeletal dysplasia. Thus, in some embodiments, a target-specific amplification primer comprises a sequence that hybridizes to a target gene or gene region that has a known association with a disease (e.g., an inherited disease).

In some embodiments, the target genes or gene regions can be analyzed for mutations, including but not limited to point mutations, single nucleotide polymorphisms, indels, gene fusions, rearrangements, alternatively spliced transcripts, or copy number variants that are associated with a disease (e.g., a cancer).

Exemplary target genes or gene regions that can be enriched for according to the methods described herein are shown in Table 1 and Table 2 below. In some embodiments, the target genes or gene regions that are enriched for are commercially available disease and cancer panels, e.g., Ion AmpliSeq™ Cancer Hotspot Panel v2 (a cancer panel targeting “hot spot” regions of 50 oncogenes and tumor suppressor genes, including coverage of KRAS, BRAF, and EGFR genes), Ion AmpliSeq™ Comprehensive Cancer Panel (a cancer panel targeting exons within >400 oncogenes and tumor suppressor genes), Ion AmpliSeq™ Inherited Disease Panel (an inherited disease panel targeting exons of over 300 genes associated with over 700 inherited diseases, including neuromuscular, cardiovascular, developmental, and metabolic diseases), and Illumina TruSeq® Amplicon Cancer Panel (a cancer panel for detecting somatic mutations across hundreds of mutational hotspots in 48 genes).

In some embodiments, a target-specific amplification primer (e.g., forward primer or reverse primer) further comprises a portion of an adapter sequence, for example as discussed above in the section “Adapters.” In some embodiments, the target-specific amplification primer comprises a portion of a P5 adapter sequence or a P7 adapter sequence. In some embodiments, the target-specific forward amplification primer comprises a portion of a P7 adapter sequence and the target-specific reverse amplification primer comprises a portion of a P5 adapter sequence. In some embodiments, the target-specific forward amplification primer comprises a portion of a P5 adapter sequence and the target-specific reverse amplification primer comprises a portion of a P7 adapter sequence. In some embodiments, a target-specific amplification primer (e.g., forward primer or reverse primer) comprises a portion of an Index 1 Read adapter sequence or Index 2 Read adapter sequence as described herein.

In some embodiments, a target-specific amplification primer comprises a portion of a P7 adapter, wherein the portion comprises at least 15, at least 20, at least 25, at least 30, or at least 35 nucleotides at the 3′ end of the P7 adapter of SEQ ID NO:4 or SEQ ID NO:6. In some embodiments, for a target-specific amplification primer, the portion of the P7 adapter is a a sequence having at least 70% identity (e.g., at least 70%, 75%, 80%, 85%, 90%, 91%, 92%, 93%, 94%, 95%, 96%, 97%, 98%, or 99% identity) to the sequence 5′-TCAGACGTGTGCTCTTCCGATCT-3′ (SEQ ID NO:8) or having the sequence of SEQ ID NO:8. In some embodiments, the target-specific amplification primer comprising the sequence of SEQ ID NO:8 is a forward amplification primer. In some embodiments, the target-specific amplification primer comprising the sequence of SEQ ID NO:8 is a reverse amplification primer. In some embodiments, the target-specific amplification primers are primers listed in Table 1 below.

In some embodiments, a target-specific amplification primer comprises a portion of a P5 adapter, wherein the portion comprises at least 15, at least 20, at least 25, at least 30, or at least 35 nucleotides at the 3′ end of the P5 adapter of SEQ ID NO:1 or SEQ ID NO:3. In some embodiments, for a target-specific amplification primer, the portion of the P5 adapter is a sequence having at least 70% identity (e.g., at least 70%, 75%, 80%, 85%, 90%, 91%, 92%, 93%, 94%, 95%, 96%, 97%, 98%, or 99% identity) to the sequence 5′-ACACTCTTTCCCTACACGACGCTCTTCCGATCT-3′ (SEQ ID NO:7) or having the sequence of SEQ ID NO:7. In some embodiments, the target-specific amplification primer comprising the sequence of SEQ ID NO:7 is a forward amplification primer. In some embodiments, the target-specific amplification primer comprising the sequence of SEQ ID NO:7 is a reverse amplification primer. In some embodiments, the target-specific amplification primers are primers listed in Table 2 below.

In some embodiments, a target-specific amplification primer comprises a portion of an Index 1 Read adapter, wherein the portion comprises at least 10, at least 15, at least 20, at least 25, or at least 30 nucleotides at the 3′ end of the Index 1 Read adapter of SEQ ID NO:109. In some embodiments, the target-specific amplification primer comprising a portion of an Index 1 Read adapter is a forward amplification primer. In some embodiments, the target-specific amplification primer comprising a portion of an Index 1 Read adapter is a reverse amplification primer.

In some embodiments, a target-specific amplification primer comprises a portion of an Index 2 Read adapter, wherein the portion comprises at least 10, at least 15, at least 20, at least 25, or at least 30 nucleotides at the 3′ end of the Index 2 Read adapter of SEQ ID NO:110. In some embodiments, the target-specific amplification primer comprising a portion of an Index 2 Read adapter is a forward amplification primer. In some embodiments, the target-specific amplification primer comprising a portion of an Index 2 Read adapter is a reverse amplification primer.

In some embodiments, the target-specific amplification primer further comprises an index or barcode sequence. In some embodiments, the index or barcode sequence is from about 4 nucleotides to about 20 nucleotides in length, about 6 nucleotides to about 12 nucleotides in length, or about 4 to about 10 nucleotides in length. In some embodiments, the index or barcode sequence is inserted between the target gene-specific sequence and the partial adapter sequence in the target-specific forward or reverse amplification primer. In some embodiments, the index or barcode sequence is inserted between the 5′-TCT-Index-ACA-3′ of the P5 adapter sequence. In some embodiments, the index or barcode sequence is inserted between the 5′-GAT-Index-GTG-3′ of the P7 adapter sequence.

Primers can be prepared by a variety of methods, including but not limited to, cloning of appropriate sequences and direct chemical synthesis using methods known in the art. See, e.g., Narang et al., Methods Enzymol 68:90 (1979). Computer programs can also be used to design primers and calculate the melting temperatures of primers. Primers can also be obtained from commercial sources, including but not limited to Integrated DNA Technologies, BioSearch Technologies, Operon Technologies, Amersham Pharmacia Biotech, Sigma, and Life Technologies.

Additional Amplification Reaction Components

For amplifying target genes or gene regions of the polynucleotide fragments by ddPCR, an amplification reaction mixture is prepared. In some embodiments, the amplification reaction mixture comprises one or more pairs of target-specific amplification primers as described herein. In some embodiments, the amplification mixture further comprises one or more of salts, nucleotides, buffers, stabilizers, DNA polymerase, a detectable agent, and nuclease-free water.

In some embodiments, the amplification reaction mixture comprises a DNA polymerase. DNA polymerases for use in the methods described herein can be any polymerase capable of replicating a DNA molecule. In some embodiments, the DNA polymerase is a thermostable polymerase. Thermostable polymerases are isolated from a wide variety of thermophilic bacteria, such as Thermus aquaticus (Taq), Pyrococcus furiosus (Pfu), Pyrococcus woesei (Pwo), Bacillus sterothermophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), as well as other species. DNA polymerases are known in the art and are commercially available. In some embodiments, the DNA polymerase is Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, or an active mutant, variant, or derivative thereof. In some embodiments, the DNA polymerase is Taq DNA polymerase. In some embodiments, the DNA polymerase is a high fidelity DNA polymerase (e.g., iProof™ High-Fidelity DNA Polymerase, Phusion® High-Fidelity DNA polymerase, Q5® High-Fidelity DNA polymerase, Platinum® Taq High Fidelity DNA polymerase, Accura® High-Fidelity Polymerase). In some embodiments, the DNA polymerase is a fast-start polymerase (e.g., FastStart™ Taq DNA polymerase or FastStart™ High Fidelity DNA polymerase).

In some embodiments, the amplification reaction mixture comprises nucleotides. Nucleotides for use in the methods described herein can be any nucleotide useful in the polymerization of a nucleic acid. Nucleotides can be naturally occurring, unusual, modified, derivative, or artificial. Nucleotides can be unlabeled, or detectably labeled by methods known in the art (e.g., using radioisotopes, vitamins, fluorescent or chemiluminescent moieties, dioxigenin). In some embodiments, the nucleotides are deoxynucleoside triphosphates (“dNTPs,” e.g., dATP, dCTP, dGTP, dTTP, dITP, dUTP, α-thio-dNITs, biotin-dUTP, fluorescein-dUTP, digoxigenin-dUTP, or 7-deaza-dGTP). dNTPs are also well known in the art and are commercially available. In some embodiments, the nucleotides do not comprise dUTP.

In some embodiments, the amplification reaction mixture comprises one or more buffers or salts. A wide variety of buffers and salt solutions and modified buffers are known in the art. For example, in some embodiments, the buffer is TRIS, TRICINE, BIS-TRICINE, HEPES, MOPS, TES, TAPS, PIPES, or CAPS. In some embodiments, the salt is potassium acetate, potassium sulfate, potassium chloride, ammonium sulfate, ammonium chloride, ammonium acetate, magnesium chloride, magnesium acetate, magnesium sulfate, manganese chloride, manganese acetate, manganese sulfate, sodium chloride, sodium acetate, lithium chloride, or lithium acetate. In some embodiments, the amplification reaction mixture comprises a salt (e.g., potassium chloride) at a concentration of about 10 mM to about 100 mM.

In some embodiments, the amplification reaction mixture comprises one or more optically detectable agents such as a fluorescent agent, phosphorescent agent, chemiluminescent agent, etc. Numerous agents (e.g., dyes, probes, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. In some embodiments, the agent is a fluorophore. A vast array of fluorophores are reported in the literature and thus known to those skilled in the art, and many are readily available from commercial suppliers to the biotechnology industry. Literature sources for fluorophores include Cardullo et al., Proc. Natl. Acad. Sci. USA 85: 8790-8794 (1988); Dexter, D. L., J. of Chemical Physics 21: 836-850 (1953); Hochstrasser et al., Biophysical Chemistry 45: 133-141 (1992); Selvin, P., Methods in Enzymology 246: 300-334 (1995); Steinberg, I. Ann. Rev. Biochem., 40: 83-114 (1971); Stryer, L. Ann. Rev. Biochem., 47: 819-846 (1978); Wang et al., Tetrahedron Letters 31: 6493-6496 (1990); Wang et al., Anal. Chem. 67: 1197-1203 (1995). Non-limiting examples of fluorophores include cyanines, fluoresceins (e.g., 5′-carboxyfluorescein (FAM), Oregon Green, and Alexa 488), HEX, rhodamines (e.g., N,N,N′,N′-tetramethyl-6-carboxyrhodamine (TAMRA), tetramethyl rhodamine, and tetramethyl rhodamine isothiocyanate (TRITC)), eosin, coumarins, pyrenes, tetrapyrroles, arylmethines, oxazines, polymer dots, and quantum dots.

In some embodiments, the detectable agent is an intercalating agent. Intercalating agents produce a signal when intercalated in double stranded nucleic acids. Exemplary intercalating agents include e.g., 9-aminoacridine, ethidium bromide, a phenanthridine dye, EvaGreen, PICO GREEN (P-7581, Molecular Probes), EB (E-8751, Sigma), propidium iodide (P-4170, Sigma), Acridine orange (A-6014, Sigma), thiazole orange, oxazole yellow, 7-aminoactinomycin D (A-1310, Molecular Probes), cyanine dyes (e.g., TOTO, YOYO, BOBO, and POPO), SYTO, SYBR Green I (U.S. Pat. No. 5,436,134: N′,N′-dimethyl-N-[4-[(E)-(3-methyl-1,3-benzothiazol-2-ylidene)methyl]-1-phenylquinolin-l-ium-2-yl]-N-propylpropane-1,3-diamine), SYBR Green II (U.S. Pat. No. 5,658,751), SYBR DX, OliGreen, CyQuant GR, SYTOX Green, SYTO9, SYTO10, SYTO17, SYBR14, FUN-1, DEAD Red, Hexidium Iodide, ethidium bromide, Dihydroethidium, Ethidium Homodimer, 9-Amino-6-Chloro-2-Methoxyacridine, DAPI, DIPI, Indole dye, Imidazole dye, Actinomycin D, Hydroxystilbamidine, LDS 751 (U.S. Pat. No. 6,210,885), and the dyes described in dyes described in Georghiou, Photochemistry and Photobiology, 26:59-68, Pergamon Press (1977); Kubota, et al., Biophys. Chem., 6:279-284 (1977); Genest, et al., Nuc. Ac. Res., 13:2603-2615 (1985); Asseline, EMBO J., 3: 795-800 (1984); Richardson, et. al., U.S. Pat. No. 4,257,774; and Letsinger, et. al., U.S. Pat. No. 4,547,569.

In some embodiments, the agent is a molecular beacon oligonucleotide probe. As described above, the “beacon probe” method relies on the use of energy transfer. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched.

In some embodiments, the agent is a radioisotope. Radioisotopes include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y.

In some embodiments, the amplification reaction mixture comprises one or more stabilizers. Stabilizers for use in the methods described herein include, but are not limited to, polyol (glycerol, threitol, etc.), a polyether including cyclic polyethers, polyethylene glycol, organic or inorganic salts, such as ammonium sulfate, sodium sulfate, sodium molybdate, sodium tungstate, organic sulfonate, etc., sugars, polyalcohols, amino acids, peptides or carboxylic acids, a quencher and/or scavenger such, as mannitol, glycerol, reduced glutathione, superoxide dismutase, bovine serum albumin (BSA) or gelatine, spermidine, dithiothreitol (or mercaptoethanol) and/or detergents such as TRITON® X-100 [Octophenol(ethyleneglycolether)], THESIT® [Polyoxyethylene 9 lauryl ether (Polidocanol C₁₂ E₉)], TWEEN® (Polyoxyethylenesorbitan monolaurate 20, NP40) and BRIJ®-35 (Polyoxyethylene23 lauryl ether).

### Multiplexing

In some embodiments, the methods described herein can be used to enrich for multiple target genes or gene regions. In some embodiments, one or more of the target genes or gene regions is a target gene or gene region described in Table 1, Table 2, or Table 4 below. In some embodiments, the target-specific amplification comprises amplifying at least 2 target genes or gene regions, at least about 5 target genes or gene regions, at least about 10 target genes or gene regions, at least about 20 target genes or gene regions, at least about 30 target genes or gene regions, at least about 40 target genes or gene regions, at least about 50 target genes or gene regions, at least about 75 target genes or gene regions, at least about 100 target genes or gene regions, at least about 200 target genes or gene regions, at least about 300 target genes or gene regions, at least about 400 target genes or gene regions, at least about 500 target genes or gene regions, at least about 1000 target genes or gene regions, at least about 1500 target genes or gene regions, at least about 2000 target genes or gene regions, at least about 2500 target genes or gene regions, at least about 3000 target genes or gene regions, at least about 4000 target genes or gene regions, or at least about 5000 target genes or gene regions (e.g., at least about 2, 3, 4, 5, 6, 7, 8, 9, 10, 15, 20, 25, 30, 35, 40, 45, 50, 60, 70, 80, 90, 100, 200, 300, 400, 500, 600, 700, 800, 900, 1000, 1500, 2000, 2500, 3000, 3500, 4000, 4500, or 5000 target genes or gene regions). In some embodiments, the target-specific amplification comprises amplifying at least about 20 target genes or gene regions (e.g., at least 20 target genes or gene regions as described in Table 1, Table 2, or Table 4 below). In some embodiments, the target-specific amplification comprises amplifying at least about 50 target genes or gene regions. In some embodiments, the target-specific amplification comprises amplifying at least about 200 target genes or gene regions. In some embodiments, the target-specific amplification comprises amplifying at least about 1000 target genes or gene regions.

Thus, in some embodiments, an amplification reaction mixture comprises multiple pairs of target-specific amplification primers. In some embodiments, the amplification reaction mixture comprises at least about 2, 5, 10, 20, 30, 40, 50, 75, 100, 200, 300, 400, 500, 1000, 1500, 2000, 2500, 3000, 3500, 4000, 4500, or 5000 pairs of target-specific amplification primers. In some embodiments, at least about 50 pairs of target-specific amplification primers are used. In some embodiments, at least about 200 pairs of target-specific amplification primers are used. In some embodiments, at least about 1000 pairs of target-specific amplification primers are used.

### Partitioning

The polynucleotide fragments comprising the target gene sequences to be amplified, and the ddPCR amplification reaction components (e.g., primers, DNA polymerase, nucleotides, buffers, salts, etc.) are partitioned into a plurality of partitions. Partitions can include any of a number of types of partitions, including solid partitions (e.g., wells or tubes) and fluid partitions (e.g., aqueous droplets within an oil phase). In some embodiments, the partitions are droplets. In some embodiments, the partitions are microchannels. Methods and compositions for partitioning a sample are described, for example, in published patent applications WO 2010/036352, US 2010/0173394, US 2011/0092373, WO 2011/120024, and US 2011/0092376, the entire content of each of which is incorporated by reference herein.

In some embodiments, the polynucleotide fragments and ddPCR reaction components are partitioned into a plurality of droplets. In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%, 6%, 7%, 8%, 9%, or 10% of droplets generated from a sample coalesce with other droplets. The emulsions can also have limited flocculation, a process by which the dispersed phase comes out of suspension in flakes. Methods of emulsion formation are described, for example, in published patent applications WO 2011/109546 and WO 2012/061444, the entire content of each of which is incorporated by reference herein.

In some embodiments, the droplet is formed by flowing an oil phase through an aqueous sample comprising the polynucleotide fragments and ddPCR reaction components. The oil phase may comprise a fluorinated base oil which may additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.75%, or 3.0% (w/w). In some embodiments, 1H,1H,2H,2H-Perfluorodecanol is added to a concentration of about 0.18% (w/w).

In some embodiments, the emulsion is formulated to produce highly monodisperse droplets having a liquid-like interfacial film that can be converted by heating into microcapsules having a solid-like interfacial film; such microcapsules may behave as bioreactors able to retain their contents through an incubation period. The conversion to microcapsule form may occur upon heating. For example, such conversion may occur at a temperature of greater than about 40°, 50°, 60°, 70°, 80°, 90°, or 95° C. During the heating process, a fluid or mineral oil overlay may be used to prevent evaporation. Excess continuous phase oil may or may not be removed prior to heating. The biocompatible capsules may be resistant to coalescence and/or flocculation across a wide range of thermal and mechanical processing. Following conversion, the microcapsules may be stored at about −70°, −20°, 0°, 3°, 4°, 5°, 6°, 7°, 8°, 9°, 10°, 15°, 20°, 25°, 30°, 35°, or 40° C.

The microcapsule partitions, which may contain one or more polynucleotide sequences and/or one or more one or more sets of primers pairs, may resist coalescence, particularly at high temperatures. Accordingly, the capsules can be incubated at a very high density (e.g., number of partitions per unit volume). In some embodiments, greater than 100,000, 500,000, 1,000,000, 1,500,000, 2,000,000, 2,500,000, 5,000,000, or 10,000,000 partitions may be incubated per mL. In some embodiments, the sample-probe incubations occur in a single well, e.g., a well of a microtiter plate, without inter-mixing between partitions. The microcapsules may also contain other components necessary for the incubation.

In some embodiments, a sample (e.g., a sample comprising polynucleotide fragments and/or ddPCR reaction components) is partitioned into at least 500 partitions, at least 1000 partitions, at least 2000 partitions, at least 3000 partitions, at least 4000 partitions, at least 5000 partitions, at least 6000 partitions, at least 7000 partitions, at least 8000 partitions, at least 10,000 partitions, at least 15,000 partitions, at least 20,000 partitions, at least 30,000 partitions, at least 40,000 partitions, at least 50,000 partitions, at least 60,000 partitions, at least 70,000 partitions, at least 80,000 partitions, at least 90,000 partitions, at least 100,000 partitions, at least 200,000 partitions, at least 300,000 partitions, at least 400,000 partitions, at least 500,000 partitions, at least 600,000 partitions, at least 700,000 partitions, at least 800,000 partitions, at least 900,000 partitions, at least 1,000,000 partitions, at least 2,000,000 partitions, at least 3,000,000 partitions, at least 4,000,000 partitions, at least 5,000,000 partitions, at least 10,000,000 partitions, at least 20,000,000 partitions, at least 30,000,000 partitions, at least 40,000,000 partitions, at least 50,000,000 partitions, at least 60,000,000 partitions, at least 70,000,000 partitions, at least 80,000,000 partitions, at least 90,000,000 partitions, at least 100,000,000 partitions, at least 150,000,000 partitions, or at least 200,000,000 partitions.

In some embodiments, a sample (e.g., a sample comprising polynucleotide fragments and/or ddPCR reaction components) is partitioned into a sufficient number of partitions such that at least a majority of partitions have at least about 0.1 but no more than about 10 targets per partition (e.g., about 0.1, 0.2, 0.3, 0.4, 0.5, 1, 2, 3, 4, 5, 6, 7, 8, 9, or 10 targets per partition). In some embodiments, at least a majority of the partitions have at least about 0.1 but no more than about 5 targets per partition (e.g., about 0.1, 0.2, 0.3, 0.4, 0.5, 1, 2, 3, 4, or 5 targets per partition). In some embodiments, at least a majority of partitions have at least about 1 but no more than about 5 targets per partition (e.g., about 1, 2, 3, 4, or 5 targets per partition). In some embodiments, on average no more than 10 targets are present in each partition. In some embodiments, on average at least about 0.1 but no more than about 10 targets are present in each partition. In some embodiments, on average at least about 1 but no more than about 5 targets are present in each partition. In some embodiments, on average about 0.1, 0.2, 0.3, 0.4, 0.5, 1, 2, 3, 4, 5, 6, 7, 8, 9, or 10 targets are present in each partition.

In some embodiments, the droplets that are generated are substantially uniform in shape and/or size. For example, in some embodiments, the droplets are substantially uniform in average diameter. In some embodiments, the droplets that are generated have an average diameter of about 0.001 microns, about 0.005 microns, about 0.01 microns, about 0.05 microns, about 0.1 microns, about 0.5 microns, about 1 microns, about 5 microns, about 10 microns, about 20 microns, about 30 microns, about 40 microns, about 50 microns, about 60 microns, about 70 microns, about 80 microns, about 90 microns, about 100 microns, about 150 microns, about 200 microns, about 300 microns, about 400 microns, about 500 microns, about 600 microns, about 700 microns, about 800 microns, about 900 microns, or about 1000 microns. In some embodiments, the droplets that are generated have an average diameter of less than about 1000 microns, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, or less than about 25 microns. In some embodiments, the droplets that are generated are non-uniform in shape and/or size.

In some embodiments, the droplets that are generated are substantially uniform in volume. For example, in some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, or about 50 nL. In some embodiments, the droplets have an average volume of about 50 picoliters to about 2 nanoliters. In some embodiments, the droplets have an average volume of about 0.5 nanoliters to about 50 nanoliters. In some embodiments, the droplets have an average volume of about 0.5 nanoliters to about 2 nanoliters.

### Target-Specific Amplification in Partitions

In some embodiments, the methods described herein comprise a target-specific amplification step that is performed in partitions. In some embodiments, the target-specific amplification step comprises amplifying a target gene sequence of a polynucleotide fragment in a partition with one of the primer pairs in the partition, thereby generating an amplicon comprising the target gene sequence flanked on the 5′ end by the portion of the first adapter sequence and flanked on the 3′ end by the portion of the second adapter sequence. In some embodiments, amplifying the nucleic acid molecules or regions of the nucleic acid molecule comprises polymerase chain reaction (PCR), droplet digital PCR, quantitative PCR, or real-time PCR.

In some embodiments, the amplification reaction is a PCR reaction. In PCR amplification, oligonucleotide primers that are complementary to the strands of a double-stranded target sequence are annealed to their complementary sequence within the target molecule, which is denatured into single strands. The annealed primers are extended with a polymerase to form a new pair of complementary strands of the target sequence. The steps of denaturation, primer annealing, and extension can be repeated until the desired number of copies or concentration of amplified sequence is obtained. In some embodiments, the annealing temperature for the target-specific amplification reaction is from 40°-70° C.

In some embodiments, the amplification reaction is a droplet digital PCR reaction. Methods for performing PCR in droplets are described, for example, in US 2014/0162266, US 2014/0302503, and US 2015/0031034, the contents of each of which is incorporated by reference. Methods of amplification are also further discussed below in the section “Nested Amplification of Target-Specific PCR Products.”

In some embodiments, the step of amplifying a target gene sequence of a polynucleotide fragment in a partition comprises at least one cycle of amplification. In some embodiments, the step of amplifying a target gene sequence of a polynucleotide fragment in a partition comprises at least 5 cycles of amplification, at least 10 cycles of amplification, at least 15 cycles of amplification, at least 20 cycles of amplification at least 25 cycles of amplification, at least 30 cycles of amplification, at least 35 cycles of amplification, or at least 40 cycles of amplification. In some embodiments, the step of amplifying a target gene sequence of a polynucleotide fragment in a partition comprises no more than 40 cycles of amplification. In some embodiments, the step of amplifying a target gene sequence of a polynucleotide fragment in a partition comprises from 2 to 30 cycles of amplification.

In some embodiments, an amplification reaction as described herein generates an amplicon comprising the target gene sequence flanked on the 5′ end by the portion of the first adapter sequence and flanked on the 3′ end by the portion of the second adapter sequence. In some embodiments, the amplicon comprises the target gene sequence flanked on the 5′ end by a portion of a P7 adapter sequence and flanked on the 3′ end by a portion of a P5 adapter sequence. In some embodiments, the amplicon comprises the target gene sequence flanked on the 5′ end by a portion of a P5 adapter sequence and flanked on the 3′ end by a portion of a P7 adapter sequence.

### Purification of Amplicons

In some embodiments, following the target-specific amplification reaction in the partitions, the amplicons are released from the partitions. In some embodiments, the partitions (e.g., droplets) are broken to release the contents of the partitions, including the amplicons. Droplet breaking can be accomplished by any of a number of methods, including but not limited to electrical methods, mechanical agitation (e.g., mixing and/or centrifugation), and introduction of a destabilizing fluid, or combinations thereof. See, e.g., Zeng et al., Anal Chem 2011, 83:2083-2089. Methods of breaking partitions are also described, for example, in US 2013/0189700, and in Akartuna et al., 2015, Lab Chip, doi: 10.1039/c4lc01285b, incorporated by reference herein.

In some embodiments, the method comprises mixing droplets with a destabilizing fluid. In some embodiments, the destabilizing fluid is chloroform. In some embodiments, the destabilizing fluid comprises a fluorinated oil.

In some embodiments, the amplicons that are released from the partitions are purified, e.g., in order to separate the amplicons from the target-specific primers, other partition components and/or to size select amplicons having a particular size or range of sizes. In some embodiments, the amplicons are purified using solid-phase reversible immobilization (SPRI) paramagnetic bead reagents. SPRI paramagnetic bead reagents are commercially available, for example in the Agencourt AMPure XP PCR purification system or SPRIselect reagent kit (Beckman-Coulter, Brea, Calif.).

Nested Amplification of Target-Specific PCR Products

In some embodiments, a second amplification reaction is performed on the amplicon products of the target-specific amplification reaction. In some embodiments, the second amplification reaction is a “nested amplification” that amplifies the amplicons comprising the partial adapter sequences, using primer sequences comprising full-length adapter sequences or a portion of the adapter sequences (e.g., at least 10, at least 15, at least 20, at least 25, at least 30, at least 40, or at least 50 or more contiguous nucleotides of the adapter sequence, or at least 40%, 50%, at least 60%, at least 70%, at least 80%, at least 90%, or at least 95% of the length of the full-length adapter sequence). In some embodiments, the target-specific amplification reaction introduces a portion of the first adapter sequence (e.g., a P7 adapter sequence) and a portion of the second adapter sequence (e.g., a P5 adapter sequence) into the polynucleotide sequence, and the subsequent nested amplification reaction introduces the full-length first adapter sequence and second adapter sequence or a portion of the first adapter sequence and second adapter sequence that includes any portion of the adapter sequence not already introduced into the polynucleotide sequence by the target-specific amplification reaction, to generate a library of polynucleotides having the entire first adapter sequence (e.g., P7 adapter sequence) and entire second adapter sequence (e.g., P5 adapter sequence).

In some embodiments, a primer sequence comprising an adapter sequence comprises a full-length P5 adapter sequence. In some embodiments, a primer sequence comprising an adapter sequence comprises a full-length P7 adapter sequence. P5 and P7 adapter sequences are discussed above in the section “Adapters.” In some embodiments, the forward primer sequence comprises a P7 adapter sequence and the reverse primer sequence comprises a P5 adapter sequence. In some embodiments, the forward primer sequence comprises a P5 adapter sequence and the reverse primer sequence comprises a P7 adapter sequence. In some embodiments, the forward and/or reverse primer comprising a full-length adapter sequence (e.g., a full-length P5 or P7 adapter sequence) comprises a barcode sequence.

In some embodiments, the forward or reverse primer for the nested amplification reaction (also referred to herein as an “amplicon primer”) comprises a sequence having at least 70% identity (e.g., at least 70%, 75%, 80%, 85%, 90%, 91%, 92%, 93%, 94%, 95%, 96%, 97%, 98%, or 99% identity) to the P5 adapter sequence of SEQ ID NO:1 or SEQ ID NO:3. In some embodiments, the forward or reverse primer for the nested amplification reaction comprises the sequence of SEQ ID NO:1. In some embodiments, the forward or reverse primer for the nested amplification reaction comprises a sequence having at least 70% identity to SEQ ID NO:1 or SEQ ID NO:3, wherein the sequence comprises the contiguous nucleic acid sequence of SEQ ID NO:2. In some embodiments, the forward or reverse primer for the nested amplification reaction comprises a sequence having at least 70% identity (e.g., at least 70%, 75%, 80%, 85%, 90%, 91%, 92%, 93%, 94%, 95%, 96%, 97%, 98%, or 99% identity) to the P7 adapter sequence of SEQ ID NO:4 or SEQ ID NO:6. In some embodiments, the forward or reverse primer for the nested amplification reaction comprises the sequence of SEQ ID NO:4. In some embodiments, the forward or reverse primer for the nested amplification reaction comprises a sequence having at least 70% identity to SEQ ID NO:4 or SEQ ID NO:6, wherein the sequence comprises the contiguous nucleic acid sequence of SEQ ID NO:5.

In some embodiments, the forward or reverse primer for the nested amplification reaction comprises a sequence having at least 70% identity (e.g., at least 70%, 75%, 80%, 85%, 90%, 91%, 92%, 93%, 94%, 95%, 96%, 97%, 98%, or 99% identity) to, or comprising the sequence of, any of SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, or SEQ ID NO:136.

For the nested amplification reaction, in some embodiments the step of amplifying the nucleic acid molecules or regions of the nucleic acid molecule comprises polymerase chain reaction (PCR), droplet digital PCR, quantitative PCR, or real-time PCR. In some embodiments, the amplification reaction is a quantitative amplification method. Quantitative amplification methods (e.g., quantitative PCR or quantitative linear amplification) involve amplification of nucleic acid template, directly or indirectly (e.g., determining a Ct value) determining the amount of amplified DNA, and then calculating the amount of initial template based on the number of cycles of the amplification. Amplification of a DNA locus using reactions is well known (see U.S. Pat. Nos. 4,683,195 and 4,683,202; PCR PROTOCOLS: A GUIDE TO METHODS AND APPLICATIONS (Innis et al., eds, 1990)). Typically, PCR is used to amplify DNA templates. However, alternative methods of amplification have been described and can also be employed. Methods of quantitative amplification are disclosed in, e.g., U.S. Pat. Nos.

6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol Biotechnol. 20(2):163-79 (2002). Amplifications can be monitored in “real time.”

In some embodiments, quantitative amplification is based on the monitoring of the signal (e.g., fluorescence of a probe) representing copies of the template in cycles of an amplification (e.g., PCR) reaction. In the initial cycles of the PCR, a very low signal is observed because the quantity of the amplicon formed does not support a measurable signal output from the assay. After the initial cycles, as the amount of formed amplicon increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., Heid et al. Genome Methods 6:986-94 (1996) with reference to hydrolysis probes.

One method for detection of amplification products is the 5′-3′ exonuclease “hydrolysis” PCR assay (also referred to as the TaqManTm assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the TaqMan™ probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in the open conformation and fluorescence is detected, while those that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

In some embodiments, the nested amplification reaction comprises at least 1 cycle of amplification, at least 2 cycles of amplification, at least 5 cycles of amplification, at least 10 cycles of amplification. In some embodiments, the nested amplification reaction comprises at least 15 cycles of amplification, at least 20 cycles of amplification at least 25 cycles of amplification, at least 30 cycles of amplification, at least 35 cycles of amplification, or at least 40 cycles of amplification.

Following the nested amplification reaction, in some embodiments, the amplification products are purified. For example, in some embodiments, the amplification products are purified using solid-phase reversible immobilization (SPRI) paramagnetic bead reagents, e.g., using the Agencourt AMPure XP PCR purification system or SPRIselect reagent kit (Beckman-Coulter, Brea, Calif.).

### III. METHODS OF DETECTION USING TARGET-ENRICHED LIBRARIES

In some embodiments, the methods described herein can be used to generate target-enriched libraries, which can be used in downstream detection and/or analysis methods.

### Sequencing

In some embodiments, the target-enriched libraries are subjected to sequencing. Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/0841,32, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

In some embodiments, nucleotide sequencing comprises high-throughput sequencing. In high-throughput sequencing, parallel sequencing reactions using multiple templates and multiple primers allows rapid sequencing of genomes or large portions of genomes. See, e.g., WO 03/004690, WO 03/054142, WO 2004/069849, WO 2004/070005, WO 2004/070007, WO 2005/003375, WO 2000/006770, WO 2000/027521, WO 2000/058507, WO 2001/023610, WO 2001/057248, WO 2001/057249, WO 2002/061127, WO 2003/016565, WO 2003/048387, WO 2004/018497, WO 2004/018493, WO 2004/050915, WO 2004/076692, WO 2005/021786, WO 2005/047301, WO 2005/065814, WO 2005/068656, WO 2005/068089, WO 2005/078130, and Seo, et al., Proc. Natl. Acad. Sci. USA (2004) 101:5488-5493.

Typically, high throughput sequencing methods share the common feature of massively parallel, high-throughput strategies, with the goal of lower costs in comparison to older sequencing methods (See, e.g., Voelkerding et al., Clinical Chem., 55: 641-658, 2009; MacLean et al., Nature Rev. Microbiol., 7:287-296; each herein incorporated by reference in their entirety). Such methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbial., 7:287-296; U.S. Pat. Nos. 6,210,891; and 6,258,568; each herein incorporated by reference in its entirety), template DNA is fragmented, end-repaired, attached to adapters, and clonally amplified in-situ by capturing single template molecules with beads bearing oligonucleotides complementary to the adapters. Each bead bearing a single template type is compartmentalized into a water-in-oil microvesicle, and the template is clonally amplified using a technique referred to as emulsion PCR. The emulsion is disrupted after amplification and beads are deposited into individual wells of a picotiter plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55. 641-658, 2009; MacLean et al., Nature Rev. Microbial., 7:287-296; U.S. Pat. Nos. 6,833,246; 7,115,400; and 6,969,488; each herein incorporated by reference in its entirety), sequencing data are produced in the form of shorter-length reads. In this method, adapter sequences on the polynucleotides (such as the adapter sequences described herein) are used to capture the template-adapter molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides (e.g., at least 300 bp×300 bp for a total of 600 bp with The MiSeq and the v3 reagent kit), with overall output exceeding 1.5 trillion nucleotide pairs per analytical run (e.g., Illumina's HiSeq 3000/HiSeq 4000).

Sequencing nucleic acid molecules using SOLiD technology (Voelkerding et al., Clinical Chem., 55: 641-658, 2009; MacLean et al., Nature Rev. Microbial., 7:287-296; U.S. Pat. Nos. 5,912,148; and 6,130,073; each herein incorporated by reference in their entirety) also involves the use of adapter sequences on polynucleotides. Typically, the process involves fragmentation of the template, attachment of oligonucleotide adapters to the fragments, attachment of the polynucleotides comprising adapters onto beads, and clonal amplification by emulsion PCR. Following this, beads bearing template are immobilized on a derivatized surface of a glass flow-cell, and a primer complementary to the adapter oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages about 35-50 nucleotides, and overall output exceeds 4 billion bases per sequencing run.

In certain embodiments, nanopore sequencing is employed (See, e.g., Astier et al., J. Am. Chem. Soc. 2006 Feb. 8; 128(5)1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, HeliScope by Helicos BioSciences is employed (Voelkerding et al., Clinical Chem., 55. 641-658, 2009; MacLean et al., Nature Rev. Microbial, 7:287-296; U.S. Pat. Nos. 7,169,560; 7,282,337; 7,482,120; 7,501,245; 6,818,395; 6,911,345; and 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (See, e.g., Science 327(5970): 1190 (2010); U.S. Pat. Appl. Pub. Nos. 2009/0026082; 2009/0127589; 2010/0301398; 2010/0197507; 2010/0188073; and 2010/0137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers the hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

### Detection Devices

In some embodiments, a detection reagent or a detectable label can be detected using any of a variety of detector devices. Exemplary detection methods include radioactive detection, optical detection (e.g., absorbance, fluorescence, or chemiluminescence), or mass spectral detection. As a non-limiting example, a fluorescent label can be detected using a detector device equipped with a module to generate excitation light that can be absorbed by a fluorophore, as well as a module to detect light emitted by the fluorophore.

In some embodiments, detectable labels in amplification products can be can be detected in bulk. For example, partitioned samples (e.g., droplets) can be combined into one or more wells of a plate, such as a 96-well or 384-well plate, and the signal(s) (e.g., fluorescent signal(s)) can be detected using a plate reader. In some cases, barcodes can be used to maintain partitioning information after the partitions are combined.

In some embodiments, the detector further comprises handling capabilities for the partitioned samples (e.g., droplets), with individual partitioned samples entering the detector, undergoing detection, and then exiting the detector. In some embodiments, partitioned samples (e.g., droplets) can be detected serially while the partitioned samples are flowing. In some embodiments, partitioned samples (e.g., droplets) are arrayed on a surface and a detector moves relative to the surface, detecting signal(s) at each position containing a single partition. Examples of detectors are provided in WO 2010/036352, the contents of which are incorporated herein by reference. In some embodiments, detectable labels in partitioned samples can be detected serially without flowing the partitioned samples (e.g., using a chamber slide).

Following acquisition of fluorescence detection data, a general purpose computer system (referred to herein as a “host computer”) can be used to store and process the data. A computer-executable logic can be employed to perform such functions as subtraction of background signal, assignment of target and/or reference sequences, and quantification of the data. A host computer can be useful for displaying, storing, retrieving, or calculating diagnostic results from the nucleic acid detection; storing, retrieving, or calculating raw data from the nucleic acid detection; or displaying, storing, retrieving, or calculating any sample or patient information useful in the methods of the present invention.

In some embodiments, the host computer, or any other computer may be used to calculate the proportion of mutations present in a sample. For example, the proportion of mutations or sequence variants can be calculated by dividing the number of partitions in which a sequence specific detection reagent detects the mutation or sequence variant by the number of partitions in which the non-specific detection reagent detects partitions containing nucleic acid (e.g., total nucleic acid, total amplified nucleic acid, total reverse transcribed nucleic acid, total DNA, or total double stranded nucleic acid).

The host computer can be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, can be included. Where the host computer is attached to a network, the connections can be provided via any suitable transport media (e.g., wired, optical, and/or wireless media) and any suitable communication protocol (e.g., TCP/IP); the host computer can include suitable networking hardware (e.g., modem, Ethernet card, WiFi card). The host computer can implement any of a variety of operating systems, including UNIX, Linux, Microsoft Windows, MacOS, or any other operating system.

Computer code for implementing aspects of the present invention can be written in a variety of languages, including PERL, C, C++, Java, JavaScript, VBScript, AWK, or any other scripting or programming language that can be executed on the host computer or that can be compiled to execute on the host computer. Code can also be written or distributed in low level languages such as assembler languages or machine languages.

Scripts or programs incorporating various features of the present invention can be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### IV. KITS

In another aspect, kits for generating target-enriched libraries are provided. In some embodiments, a kit comprises:
(a) a first composition for partitioning into a plurality of partitions, wherein the composition comprises a plurality of primer pairs, each primer pair comprising a forward primer and a reverse primer for amplifying a target gene, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence; and
(b) a second composition comprising a first primer and a second primer, wherein the first primer comprises the first adapter sequence and the second primer comprises the second adapter sequence.

In some embodiments, the first composition comprises target-specific amplification primers as described in Section II above. In some embodiments, the target-specific amplification primers comprise partial P5 and P7 adapter sequences, or partial Index 1 Read and Index 2 Read adapter sequences. In some embodiments, the target-specific amplification primers are primers listed in Table 1 or Table 2 above.

In some embodiments, the first composition comprises primers for nested amplification as described in Section II above. In some embodiments, the second composition comprises primers comprising P5 and P7 adapter sequences. In some embodiments, the second composition comprises primers comprising Index 1 Read and Index 2 Read adapter sequences.

In some embodiments, the first composition and/or the second composition further comprises one or more reagents selected from the group consisting of salts, nucleotides, buffers, stabilizers, DNA polymerase, detectable agents, and nuclease-free water. Reagents for target-specific amplification are described in Section II above. In some embodiments, a composition comprises a master mix that can be used for generating droplets (e.g., ddPCR Supermix for probes, no dUTP (Bio-Rad, Hercules, Calif.).

In some embodiments, the kit further comprises instructions for performing a method as described herein.

### V. EXAMPLES

The following examples are offered to illustrate, but not to limit, the claimed invention.

### Example 1

### Target Enrichment for 50-Plex Cancer Panel

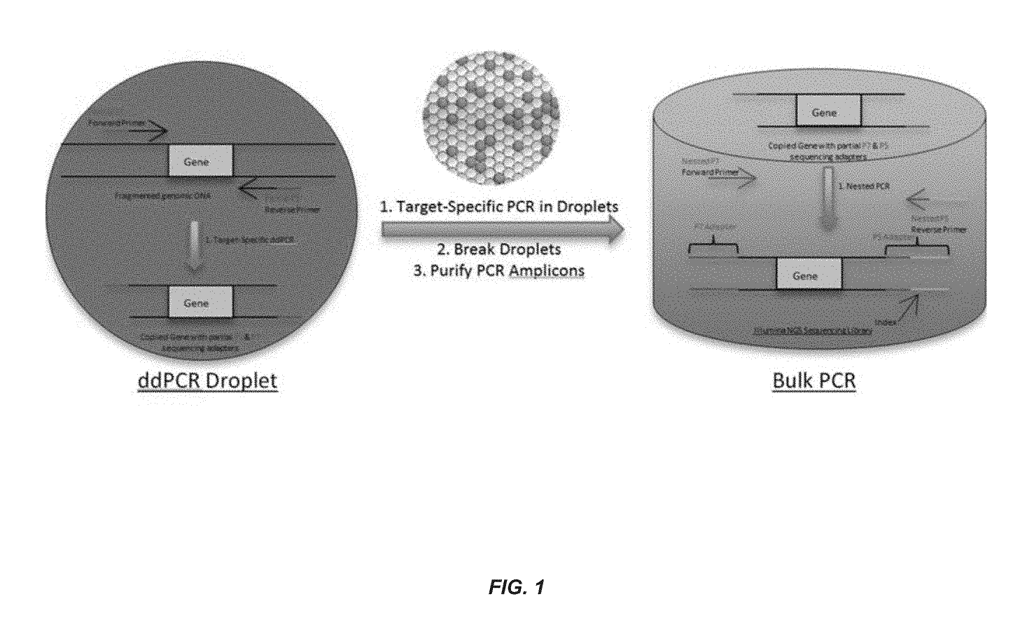

Target enrichment was performed for a 50-plex cancer panel using a target-specific, then nested PCR library construction approach, followed by droplet digital (ddPCR) and sequencing. A schematic for the target enrichment approach is shown in FIG. 1.

### Materials and Methods:

Human genomic DNA was fragmented to a median size of approximately 300 bp with NEBNext® dsDNA fragmentase (New England Biolabs, Inc., Ipswich, Mass.). Following the reaction, the fragmented DNA was purified with a 1.0× ratio of sample:Agencourt AMPure XP beads (Beckman Coulter, Brea, Calif.).

Target-specific PCR amplification reactions were run using a 50-plex of cancer target-specific forward and reverse primers having partial Illumina P5 and P7 adapter sequences, respectively. Both the bulk and ddPCR reactions used ddPCR supermix for probes, target-specific 50-plex of forward and reverse primers (starting UOM 1.0 μM each, final in reaction of 50 nM each), and EDTA-chelated fragmented reaction (starting UOM 0.64 ng/μL, final in reaction of 0.15 ng/μL).

The forward and reverse primer sequences that were used for the 50-plex are set forth in Table 1 and Table 2 below. 15 amplification cycles were performed for bulk reactions vs. droplet reactions. Following the amplification reactions, for the droplet reactions, the droplets were subjected to a droplet breaking/amplicon purification protocol with 20% perfluorobutanol/80% HFE7500. The amplicons recovered from droplets (and not for those in bulk) were subject to AMPure XP purifications at a 1.0× ratio to remove unused primers and products less than equal to 100 bp.

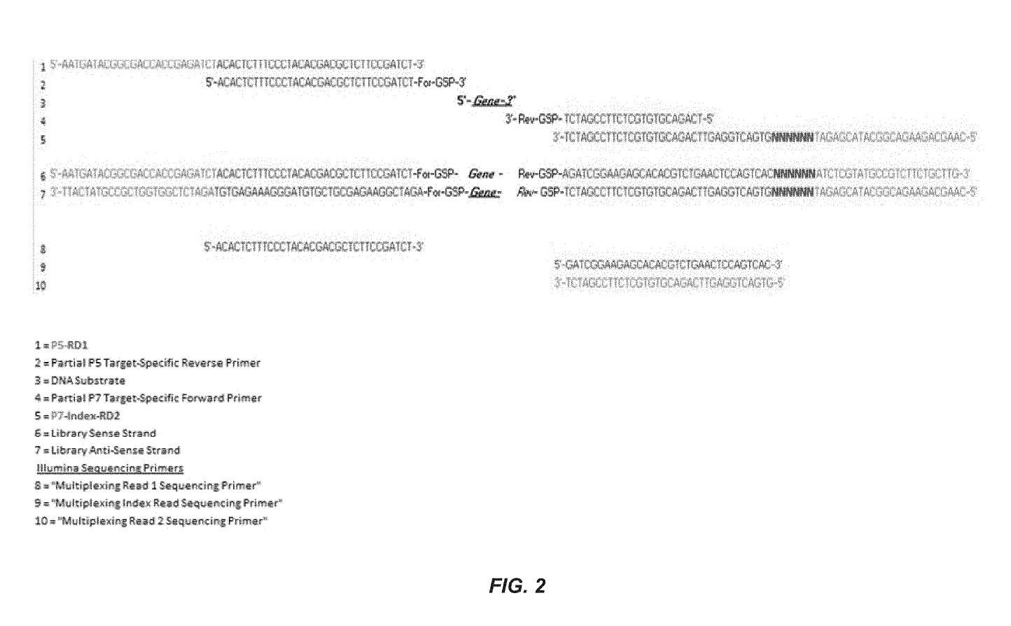

Three trials of “nested” PCR for 15 cycles each were performed, in which the remainders of the P5 and P7 Illumina adapters were incorporated to complete the sequencing libraries for each amplicon from the target-specific PCRs. See, e.g., FIG. 2. The primers that were used for the nested PCR amplification were the P5 RD1, P7 Index6 RD2, and P7 Index12 RD2 sequences set forth below:

P5 RD1:
(SEQ ID NO: 1)
AAT GAT ACG GCG ACC ACC GAG ATC TAC ACT CTT
TCC CTA CAC GAC GCT CTT CCG ATC T
P7 Index6 RD2:
(SEQ ID NO: 111)
CAAGCAGAAGACGGCATACGAGATGCCAATGTGACTGGAGTTCAGA
CGTGTGCTCTTCCGATCT
P7 Index12 RD2:
(SEQ ID NO: 112)
CAAGCAGAAGACGGCATACGAGATCTTGTAGTGACTGGAGTTCAGA
CGTGTGCTCTTCCGATCT

In trial 1, the bulk non-AMPure purified and droplet perfluorobutonol/HFE7500 AMPure purified target-specific amplicons were used. In trial 2, bulk vs. droplet perfluorobutonol/HFE7500 target-specific products that had not been subject to AMPure purifications were used for an attempt at equivalency. In trial 3, the target-specific amplicons were diluted 1/10 instead of 135.6 in an attempt at higher yields of library products.

After the nested PCR amplification reaction, the amplicons were subject to 1.0× AMPure purifications to remove undesired products less than equal to 100 bp. The Bioanalyzer (Agilent Technologies, Santa Clara, Calif.) was used to determine the sizes of the libraries. Evagreen & Taqman ddPCR were used to determine the concentrations of the amplicons at various stages in the protocol and the libraries in total, respectively. The libraries were sequenced on the Illumina MiSeq sequencer. In trial 1, it was found that libraries appeared to be present for both bulk & droplet-derived target-specific PCR materials. In trial 2, it was also found that libraries resulted from both the bulk & droplet-derived target-specific PCR materials. In trial 3, where the same procedure was followed, but with 13.56-fold more starting material in an attempt to generate more libraries, more libraries were successfully generated.

TABLE 1
50-plex Partial P7 + Forward
Gene-Specific Primer Sequences

| Assay | Gene | Oligo Name | Partial P7 + Forward Gene-Specific Primer | SEQ ID NO: |
|---|---|---|---|---|
| 1 | ABL1 | P7_part_ABL1_F | TCAGACGTGTGCTCTTCCGATCTGGAACGCACGGACAT | 9 |
| 2 | ABL1 | P7_part_ABL1_F | TCAGACGTGTGCTCTTCCGATCTCAAGCTGGGCGGG | 10 |
| 3 | AKT1 | P7_part_AKT1_F | TCAGACGTGTGCTCTTCCGATCTGAGGAGGAAGTAGCGTG | 11 |
| 4 | APC | P7_part_APC_F | TCAGACGTGTGCTCTTCCGATCTCACCCAAAAGTCCACCT | 12 |
| 5 | ATM | P7_part_ATM_F | TCAGACGTGTGCTCTTCCGATCTCAGTGAAAGATTCATCTAATGG | 13 |
| 6 | BRAF | P7_part_BRAF_F | TCAGACGTGTGCTCTTCCGATCTCAGACAACTGTTCAAACTGA | 14 |
| 7 | CDH1 | P7_part_CDH1_F | TCAGACGTGTGCTCTTCCGATCTACCTTCAATGTGTTTGGTT | 15 |
| 8 | CDKN2A | P7_part_CDKN2A_F | TCAGACGTGTGCTCTTCCGATCTGGTACCGTGCGACAT | 16 |
| 9 | CSF1R | P7_part_CSF1R_F | TCAGACGTGTGCTCTTCCGATCTCCTGTCGTCAACTCCT | 17 |
| 10 | CTNNB1 | P7_part_CTNNB1_F | TCAGACGTGTGCTCTTCCGATCTCAGTCTTACCTGGACTCTG | 18 |
| 11 | EGFR | P7_part_EGFR_F | TCAGACGTGTGCTCTTCCGATCTGCAGCATGTCAAGATCAC | 19 |
| 12 | ERBB2 | P7_part_ERBB2_F | TCAGACGTGTGCTCTTCCGATCTGAGAATGTGAAAATTCCAGTG | 20 |
| 13 | ERBB4 | P7_part_ERBB4_F | TCAGACGTGTGCTCTTCCGATCTGCATATTTGCCATTTTGGAT | 21 |
| 14 | FBXW7 | P7_part_FBXW7_F | TCAGACGTGTGCTCTTCCGATCTTGACAAGATTTTCCCTTACC | 22 |
| 15 | FGFR1 | P7_part_FGFR1_F | TCAGACGTGTGCTCTTCCGATCTCACGCATACGGTTTGG | 23 |
| 16 | FGFR2 | P7_part_FGFR2_F | TCAGACGTGTGCTCTTCCGATCTCAGTCCGGCTTGGAG | 24 |
| 17 | FGFR3 | P7_part_FGFR3_F | TCAGACGTGTGCTCTTCCGATCTAGGAGCTGGTGGAGG | 25 |
| 18 | FLT3 | P7_part_FLT3_F | TCAGACGTGTGCTCTTCCGATCTTGACAACATAGTTGGAATCAC | 26 |
| 19 | GNA11 | P7_part_GNA11_F | TCAGACGTGTGCTCTTCCGATCTCTGTGTCCTTTCAGGATG | 27 |
| 20 | GNAQ | P7_part_GNAQ_F | TCAGACGTGTGCTCTTCCGATCTAGCAGTGTATCCATTTTCTT | 28 |
| 21 | GNAS | P7_part_GNAS_F | TCAGACGTGTGCTCTTCCGATCTGACCTCAATTTTGTTTCAGG | 29 |
| 22 | HNF1A | P7_part_HNF1A_F | TCAGACGTGTGCTCTTCCGATCTTACCAACCAAGAAGGGG | 30 |
| 23 | HRAS | P7_part_HRAS_F | TCAGACGTGTGCTCTTCCGATCTATGGTCAGCGCACTC | 31 |
| 24 | IDH1 | P7_part_IDH1_F | TCAGACGTGTGCTCTTCCGATCTAACATGACTTACTTGATCCC | 32 |
| 25 | JAK2 | P7_part_JAK2_F | TCAGACGTGTGCTCTTCCGATCTCACAAGCATTTGGTTTTAAATTAT | 33 |
| 26 | JAK3 | P7_part_JAK3_F | TCAGACGTGTGCTCTTCCGATCTCTCTTACCCACTCCAGG | 34 |
| 27 | KDR | P7_part_KDR_F | TCAGACGTGTGCTCTTCCGATCTAGTCAGGCTGGAGAATC | 35 |
| 28 | KIT | P7_part_KIT_F | TCAGACGTGTGCTCTTCCGATCTCCTTACTCATGGTCGGAT | 36 |
| 29 | KRAS | P7_part_KRAS_F | TCAGACGTGTGCTCTTCCGATCTGTATCGTCAAGGCACTCT | 37 |
| 30 | MET | P7_part_MET_F | TCAGACGTGTGCTCTTCCGATCTGTTGCTGATTTTGGTCTTG | 38 |
| 31 | MLH1 | P7_part_MLH1_F | TCAGACGTGTGCTCTTCCGATCTACAATATTCGCTCCATCTTT | 39 |
| 32 | MPL | P7_part_MPL_F | TCAGACGTGTGCTCTTCCGATCTTCAGCGCCGTCCT | 40 |
| 33 | NOTCH1 | P7_part_NOTCH1_F | TCAGACGTGTGCTCTTCCGATCTCGAGCTGGACCACTG | 41 |
| 34 | NPM1 | P7_part_NPM1_F | TCAGACGTGTGCTCTTCCGATCTATGTCTATGAAGTGTTGTGG | 42 |
| 35 | NRAS | P7_part_NRAS_F | TCAGACGTGTGCTCTTCCGATCTCATGTATTGGTCTCTCATGG | 43 |
| 36 | PDGFRA | P7_part_PDGFRA_F | TCAGACGTGTGCTCTTCCGATCTTGTGAAGATCTGTGACTTTG | 44 |
| 37 | PIK3CA | P7_part_PIK3CA_F | TCAGACGTGTGCTCTTCCGATCTACAATCTTTTGATGACATTGC | 45 |
| 38 | PTEN | P7_part_PTEN_F | TCAGACGTGTGCTCTTCCGATCTATTTAACCATGCAGATCCTC | 46 |
| 39 | PTPN11 | P7_part_PTPN11_F | TCAGACGTGTGCTCTTCCGATCTTTCATGATGTTTCCTTCGTA | 47 |
| 40 | RB1 | P7_part_RB1_F | TCAGACGTGTGCTCTTCCGATCTCCCTACCTTGTCACCAAT | 48 |
| 41 | RET | P7_part_RET_F | TCAGACGTGTGCTCTTCCGATCTCACCCACAGATCCACTG | 49 |
| 42 | SMAD4 | P7_part_SMAD4_F | TCAGACGTGTGCTCTTCCGATCTTACTCAGGATGAGTTTTGTG | 50 |
| 43 | SMARCB1 | P7_part_SMARCB1_F | TCAGACGTGTGCTCTTCCGATCTTCTGTACAAGAGATACCCC | 51 |
| 44 | SMO | P7_part_SMO_F | TCAGACGTGTGCTCTTCCGATCTATGTTTGGAACTGGCATC | 52 |
| 45 | STK11 | P7_part_STK11_F | TCAGACGTGTGCTCTTCCGATCTGCGCGGACGAGGA | 53 |
| 46 | TP53 | P7_part_TP53_F | TCAGACGTGTGCTCTTCCGATCTCGCAAATTTCCTTCCACT | 54 |
| 47 | VHL | P7_part_VHL_F | TCAGACGTGTGCTCTTCCGATCTCTTTGCTTGTCCCGATAG | 55 |
| 48 | BRAF | P7_part_BRAF_F | TCAGACGTGTGCTCTTCCGATCTTGGAAAAATAGCCTCAATTCT | 56 |
| 49 | PIK3CA | P7_part_PIK3CA_F | TCAGACGTGTGCTCTTCCGATCTAGTAATTGAACCAGTAGGC | 57 |
| 50 | EGFR | P7_part_EGFR_F | TCAGACGTGTGCTCTTCCGATCTAAGGAAACTGAATTCAAAAAGA | 58 |

TABLE 2
50-plex Partial P5 + Reverse
Gene-Specific Primer Sequences

| Assay | Gene | Oligo Name | Partial P5 + Reverse Gene-Specific Primer | SEQ ID NO |
|---|---|---|---|---|
| 1 | ABL1 | P5_part_ABL1_R | ACACTCTTTCCCTACACGACGCTCTTCCGATCTCACGGCCACCGTC | 59 |
| 2 | ABL1 | P5_part_ABL1_R | ACACTCTTTCCCTACACGACGCTCTTCCGATCTCAGGCTGTATTTCTTCCAC | 60 |
| 3 | AKT1 | P5_part_AKT1_R | ACACTCTTTCCCTACACGACGCTCTTCCGATCTTCTCACCACCCGCA | 61 |
| 4 | APC | P5_part_APC_R | ACACTCTTTCCCTACACGACGCTCTTCCGATCTAGAAGTACATCTGCTAAACAT | 62 |
| 5 | ATM | P5_part_ATM_R | ACACTCTTTCCCTACACGACGCTCTTCCGATCTCAGAAAGAATGTCTTTGAGTAG | 63 |
| 6 | BRAF | P5_part_BRAF_R | ACACTCTTTCCCTACACGACGCTCTTCCGATCTCATGAAGACCTCACAGTAAA | 64 |
| 7 | CDH1 | P5_part_CDH1_R | ACACTCTTTCCCTACACGACGCTCTTCCGATCTGTTATGGAACTGCTCACC | 65 |
| 8 | CDKN2A | P5_part_CDKN2A_R | ACACTCTTTCCCTACACGACGCTCTTCCGATCTACGTGCGCGATGC | 66 |
| 9 | CSF1R | P5_part_CSF1R_R | ACACTCTTTCCCTACACGACGCTCTTCCGATCTGGATATCGCCCAGCC | 67 |
| 10 | CTNNB1 | P5_part_CTNNB1_R | ACACTCTTTCCCTACACGACGCTCTTCCGATCTTTACCACTCAGAGAAGGAG | 68 |
| 11 | EGFR | P5_part_EGFR_R | ACACTCTTTCCCTACACGACGCTCTTCCGATCTTTCTGCATGGTATTCTTTCTC | 69 |
| 12 | ERBB2 | P5_part_ERBB2_R | ACACTCTTTCCCTACACGACGCTCTTCCGATCTTTGTTGGCTTTGGGGG | 70 |
| 13 | ERBB4 | P5_part_ERBB4_R | ACACTCTTTCCCTACACGACGCTCTTCCGATCTAAAGATGGAAACTTTGGACT | 71 |
| 14 | FBXW7 | P5_part_FBXW7_R | ACACTCTTTCCCTACACGACGCTCTTCCGATCTGTATACACACCTTATATGGGC | 72 |
| 15 | FGFR1 | P5_part_FGFR1_R | ACACTCTTTCCCTACACGACGCTCTTCCGATCTCATAGATGCTCTCCCCTC | 73 |
| 16 | FGFR2 | P5_part_FGFR2_R | ACACTCTTTCCCTACACGACGCTCTTCCGATCTCTCCTTTCTTCCCTCTCTC | 74 |
| 17 | FGFR3 | P5_part_FGFR3_R | ACACTCTTTCCCTACACGACGCTCTTCCGATCTGTAGCTGAGGATGCCTG | 75 |
| 18 | FLT3 | P5_part_FLT3_R | ACACTCTTTCCCTACACGACGCTCTTCCGATCTAAAGTGGTGAAGATATGTGAC | 76 |
| 19 | GNA11 | P5_part_GNA11_R | ACACTCTTTCCCTACACGACGCTCTTCCGATCTTGGATCCACTTCCTCC | 77 |
| 20 | GNAQ | P5_part_GNAQ_R | ACACTCTTTCCCTACACGACGCTCTTCCGATCTTTAACCTTGCAGAATGGTC | 78 |
| 21 | GNAS | P5_part_GNAS_R | ACACTCTTTCCCTACACGACGCTCTTCCGATCTACTTGGTCTCAAAGATTCC | 79 |
| 22 | HNF1A | P5_part_HNF1A_R | ACACTCTTTCCCTACACGACGCTCTTCCGATCTCCTGGAACAGGATCTGC | 80 |
| 23 | HRAS | P5_part_HRAS_R | ACACTCTTTCCCTACACGACGCTCTTCCGATCTGATGACGGAATATAAGCTGG | 81 |
| 24 | IDH1 | P5_part_IDH1_R | ACACTCTTTCCCTACACGACGCTCTTCCGATCTAGTGGATGGGTAAAACCTA | 82 |
| 25 | JAK2 | P5_part_JAK2_R | ACACTCTTTCCCTACACGACGCTCTTCCGATCTAAAGCCTGTAGTTTTACTTACT | 83 |
| 26 | JAK3 | P5_part_JAK3_R | ACACTCTTTCCCTACACGACGCTCTTCCGATCTCAGCCCCAATCCCAATA | 84 |
| 27 | KDR | P5_part_KDR_R | ACACTCTTTCCCTACACGACGCTCTTCCGATCTGCAGAACTTTTAAAGCTGAT | 85 |
| 28 | KIT | P5_part_KIT_R | ACACTCTTTCCCTACACGACGCTCTTCCGATCTGGGTACTCACGTTTCCTT | 86 |
| 29 | KRAS | P5_part_KRAS_R | ACACTCTTTCCCTACACGACGCTCTTCCGATCTTATTTTTATTATAAGGCCTGCTG | 87 |
| 30 | MET | P5_part_MET_R | ACACTCTTTCCCTACACGACGCTCTTCCGATCTCAGCTTTGCACCTGTTT | 88 |
| 31 | MLH1 | P5_part_MLH1_R | ACACTCTTTCCCTACACGACGCTCTTCCGATCTTGATGGAATGATAAACCAAGA | 89 |
| 32 | MPL | P5_part_MPL_R | ACACTCTTTCCCTACACGACGCTCTTCCGATCTGGGCGGTACCTGTAGT | 90 |
| 33 | NOTCH1 | P5_part_NOTCH1_R | ACACTCTTTCCCTACACGACGCTCTTCCGATCTTACAGGTGCCTGAGCA | 91 |
| 34 | NPM1 | P5_part_NPM1_R | ACACTCTTTCCCTACACGACGCTCTTCCGATCTGAAATAAGACGGAAAATTTTTTAAC | 92 |
| 35 | NRAS | P5_part_NRAS_R | ACACTCTTTCCCTACACGACGCTCTTCCGATCTGTTTGTTGGACATACTGGAT | 93 |
| 36 | PDGFRA | P5_part_PDGFRA_R | ACACTCTTTCCCTACACGACGCTCTTCCGATCTGCCTTTCGACACATAGTTC | 94 |
| 37 | PIK3CA | P5_part_PIK3CA_R | ACACTCTTTCCCTACACGACGCTCTTCCGATCTAAGCCTCTTGCTCAGTT | 95 |
| 38 | PTEN | P5_part_PTEN_R | ACACTCTTTCCCTACACGACGCTCTTCCGATCTTGAGGGAACTCAAAGTACA | 96 |
| 39 | PTPN11 | P5_part_PTPN11_R | ACACTCTTTCCCTACACGACGCTCTTCCGATCTGATAAATCGGTACTGTGCTT | 97 |
| 40 | RB1 | P5_part_RB1_R | ACACTCTTTCCCTACACGACGCTCTTCCGATCTAATCCGTAAGGGTGAACTA | 98 |
| 41 | RET | P5_part_RET_R | ACACTCTTTCCCTACACGACGCTCTTCCGATCTGGAGAAGAGGACAGCG | 99 |
| 42 | SMAD4 | P5_part_SMAD4_R | ACACTCTTTCCCTACACGACGCTCTTCCGATCTTCAATCCAGCAAGGTGT | 100 |
| 43 | SMARCB1 | P5_part_SMARCB1_R | ACACTCTTTCCCTACACGACGCTCTTCCGATCTTGCAACTATTTTCTTCCTCT | 101 |
| 44 | SMO | P5_part_SMO_R | ACACTCTTTCCCTACACGACGCTCTTCCGATCTGTACGCCTCCAGATGAG | 102 |
| 45 | STK11 | P5_part_STK11_R | ACACTCTTTCCCTACACGACGCTCTTCCGATCTGAAGTCCTGAGTGTAGATGA | 103 |
| 46 | TP53 | P5_part_TP53_R | ACACTCTTTCCCTACACGACGCTCTTCCGATCTCCTCACTGATTGCTCTTAG | 104 |
| 47 | VHL | P5_part_VHL_R | ACACTCTTTCCCTACACGACGCTCTTCCGATCTAGAAGCCCATCGTGTG | 105 |
| 48 | BRAF | P5_part_BRAF_R | ACACTCTTTCCCTACACGACGCTCTTCCGATCTGGGTCCCATCAGTTTGA | 106 |
| 49 | PIK3CA | P5_part_PIK3CA_R | ACACTCTTTCCCTACACGACGCTCTTCCGATCTCTTTATGGTTATTTGCATTTTAGA | 107 |
| 50 | EGFR | P5_part_EGFR_R | ACACTCTTTCCCTACACGACGCTCTTCCGATCTACCTTATACACCGTGCC | 108 |

### Example 2

### Target Enrichment of Multiplexed Panel Assays in Droplets Improves NGS Library Construction

Droplet Digital PCR (ddPCR™) reduces biases and improves representation of amplicons in next-generation sequencing (NGS) libraries. The amplicons generated by multiplexing assays are improved when partitioned, compared with standard single-tube multiplex NGS methods. Partitioning the sample into droplets reduces biases that arise in PCR such as competition between assays. Custom multiplexed assays were tested for improvements in read coverage when comparing standard workflows and Droplet Digital PCR. Here we present a facile methodology which easily integrates into current NGS amplicon library workflows for improvement in reducing amplification bias in multiplex amplicon panels containing cancer, microbial, or viral targets.

### Materials and Methods:

Human genomic DNA (Coriell DNA NA18853) was subjected to Covaris shearing to produce 300 bp average fragement sized DNA. A broad panel of 200 PCR assays generating amplicons targeting genes ranging in size from 60 bp to 200 bp and GC content ranging from 25.4% to 76.9% was tested for multiplexing. This 200-plex utilized PrimePCRTM custom assays (50 nM each, Bio-Rad); all the genes are listed in the custom 200-plex supplementary table. ddPCR supermix for probes (no dUTP) (Bio-Rad, #186-3023) was used except where noted. Additional Potassium Chloride (Ambion™ 2M KCl, #AM9640G) was added to improve multiplexing in droplets to a final concentration of 40mM. Droplets were generated on the QX200™ Droplet Generator instrument (Bio-Rad, #186-4002) using DG8™ Cartridges for QX200™/QX100™ Droplet Generator (Bio-Rad #186-4008) and the amplification reaction setup scheme listed in Table 3 below (40 cycles). Droplets were transferred to Eppendorf® twin.tec semi-skirted 96-well plate, the plate was sealed using the Bio-Rad PX1™ PCR plate sealer (#181-4000) with Pierceable Foil Heat Seal—(Bio-Rad #181-4040) and thermal cycling was performed on a Bio-Rad C1000TM thermal cycler (#185-1196) as follows: 95° C. for 10 min (1 cycle); 10 to 40 cycles of: 94° C. for 30 sec, 50° C. for 30 sec, 68° C. for 1 min; hold at 4° C. Droplets were recovered according to the following protocol:
1. Pipet out the entire volume of droplets and oil from a well into a 1.5mL tube (Combine replicate wells if desired)
2. Pipet and discard the bottom oil phase after the droplets float to the top of the tube
3. Add 20 μL low TE for each well used, add additional TE by multiplying the number of combined replicate wells if applicable
4. In a fume hood add 70 μL of chloroform for each well and cap the tube, add additional chloroform multiplying the number of combined replicate wells if applicable
5. Vortex the tube at maximum speed for 1 minute
6. Centrifuge at 15,500 g for 10 minutes
7. Carefully remove the upper aqueous phase by pipetting, avoiding the chloroform phase (lower phase), and transfer the aqueous phase to a new 1.5 mL tube
8. Dispose of chloroform phase appropriately

The aqueous phase recovered from droplets contains recovered DNA, dNTPs, primers. If desired, visualize products on an Experion 1K DNA chip and/or make 10-fold dilution series and re-quantify the products using ddPCR.

Amplicons were adapted with TruSeq sequencing adapters according to the illumina TrusSeq LT protocol. The libraries generated were indexed according to the type of multiplex amplification method used in order to compare “bulk” vs. “droplet” generated libraries in the same sequencing run. Libraries were quantified using ddPCR™ Library Quantification Kit for Illumina TruSeq (Bio-Rad, #186-3040) in order to obtain equal representation of the pooled libraries and maximize the loading of the sequencer (approximately +/−15% difference between total reads of each indexed library). Sequencing was performed using an illumina MiSeq sequencer with MiSeq Reagent Kit v2 sequencing reagents. Amplicons products were also visualized on an Experion™ automated electrophoresis station (Bio-Rad) for comparison of the quality of the amplication method used in “bulk” vs. “droplet.”

TABLE 3
Amplification Reaction Setup

| Component | μL | Final concentration |
|---|---|---|
| 2x Droplet PCR Supermix for Probes (no | 10 | 1x |
| dUTP) | | |
| 200plex primers @ 250 nM each | 5 | 50 nM each |
| Sheared DNA ~300 bp (1.67 ng/μL) | 1 | 2.5 TPD (targets |
| | | per droplet) |
| 2M KCl | 0.4 | 40 mM |
| Water | 3.6 | q.s. |
| Final volume | 20 | |

### Results and Discussion:

Targeted panels are of increasing importance for NGS applications as they can yield specific information at great sequencing depth. One concern for NGS applications is the PCR bias inherently introduced by the high multiplex. Here we demonstrate reduced amplification by making use of the power of droplet partitioning. Droplet partitioning reduces bias by utilizing low target template occupancy in droplets whilst having all primer pairs of the multiplex being equally represented in the droplets. This affords a reduction in PCR amplification bias by significantly reducing the number of competing PCR reactions in each partition. This gives the less efficient PCR target amplicons opportunity to amplify an hence provides a more uniform representation of the amplicons which were amplified in droplets as compared with a traditional single tube bulk PCR reaction where all amplicons are mutually competing for resources in the PCR reaction.

Table 4 is a list of the genes used in the 200-plex to demonstrate the power of partitioning in droplets prior to amplification. 200 genes were randomly selected and tested in droplets versus bulk reactions, then TruSeq LT library preparation was conducted on the samples after 40 cycles of PCR according to the conditions described above. 40 cycles was performed in order to visualize on Experion gel, although the number of cycles may be varied depending on starting input DNA amount and library preparation methodology used. Total DNA (Coriell institute NA18853) input was lOng of Covaris sheared DNA with an average fragmentation of 300 bp. A total of 6 wells were used to distribute the lOng of DNA which contained approximately 600,000 targets of the 200plex investigated (3030.3 Genomic Equivalents*200=606,060 total targets in a reaction). This concentration of targets is approximately 5 Targets Per Droplet (TPD) (600,000 targets/(6 wells*20,000 droplets/well=5 TPD)). The droplet reaction and bulk reactions were identical and setup according to the conditions in Table 3. We empirically found the addition of KCl in the amount found in Table 3 was helpful to the multiplex in droplets, as well as the 3-step cycling conditions, where the anneal temperature was 10° C. lower than the average anneal temperature of the primers. For example, if the average Tm of the primers in the multiplex is 60° C., then it may be beneficial to run the annealing temperature during thermal cycling at 50° C.

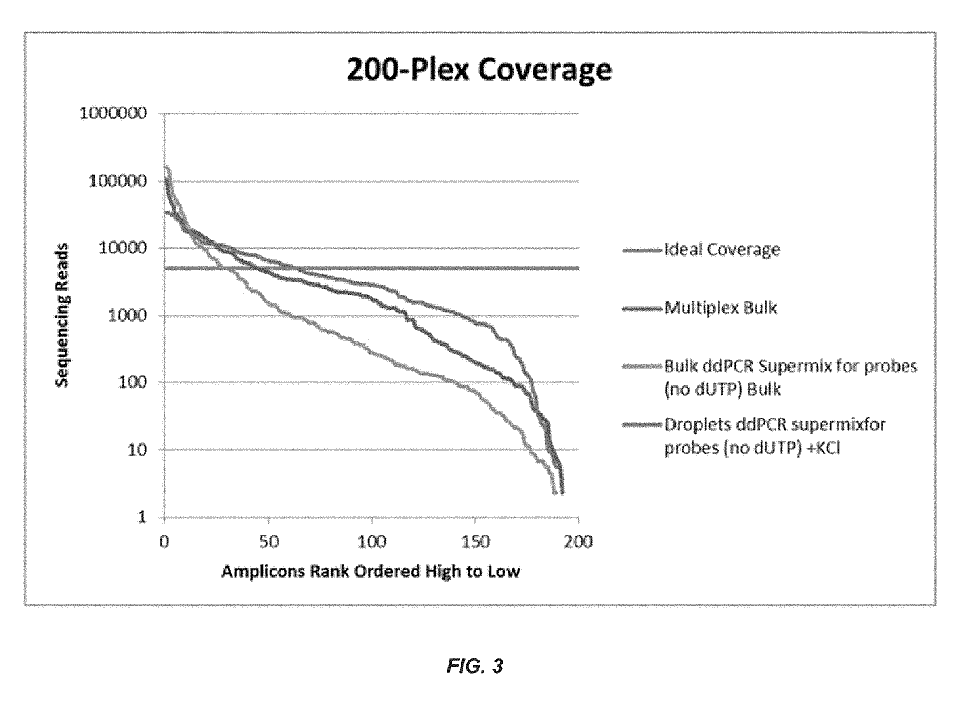

FIG. 3 clearly demonstrates the power of partitioning of the 200plex primer pairs when used in droplets compared with a single bulk PCR amplification reaction. The partitioned reaction has improved uniformity of the number of reads per target amplicon compared with the bulk reaction. The samples were indexed using illumina TruSeq LT workflow so that droplet and bulk could be assessed in the same sequencing run on an illumina MiSeq Sequencer. Note that the y-axis is the number of reads per amplicon is a base-10 log scale, therefore small changes are significant improvements in uniformity. The blue line represents the theoretical ideal distribution of the sequencing reads, where each amplicon is amplified 100% efficiently. The green line is data representing the sequencing reads from amplification performed in droplets. The orange line is the same master mix used in the droplet amplified case, with the exception of using it in a bulk reaction (no partitioning). The red line is the trace of the sequencing reads from a bulk master mix designed for high multiplexing from vendor “A.” All of the data was acquired in the same sequencing run by using unique index tags to distinguish which reads came from which amplification method used. The reads are rank ordered by the amplicons receiving the highest number of reads to the lowest number of reads on the x-axis. Clearly the droplet partitioned reaction improves the uniformity of sequencing reads per amplicon as compared to the bulk reactions. This occurs over the vast majority of amplicons tested. By randomly selecting a 200plex without bioinformatically or empirically predetermining if the amplicons would amplify well together, this experiment suggests that partitioning in general assists in improving amplification bias compared with bulk reactions. Commercial targeted panels which have been thoroughly vetted for performance should also be improved. One can also imagine utilizing this droplet PCR technique with primers which bear the sequencing oligonucleotide adapters already incorporated in the primers in order to streamline NGS library construction.

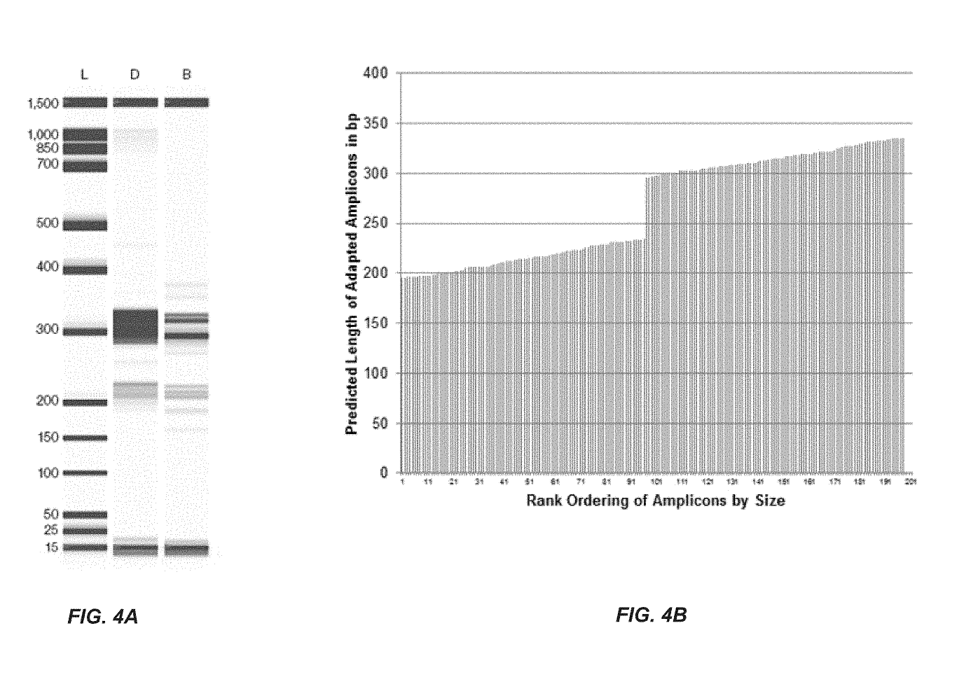

FIG. 4A is an Experion Gel of the 200plex recovered material. The material was gathered from recovered amplification of droplets and bulk reactions. FIG. 4B shows that there are 2 size populations expected for the library inserts (with adapters) which range from approximately 200 bp-225 bp and the second population ranging from 300 bp-335 bp. Note that in droplets on the Experion gel in FIG. 4A, the two populations (with TruSeq adapters) is more uniform and has less off-target bands compared to the bulk reaction which has more off-target, potentially chimeric, amplifications.

TABLE 4
Genes used in 200-plex

| | | Amp | | | Amp | | | Amp |
|---|---|---|---|---|---|---|---|---|
| | | Length | | | Length | | | Length |
| Ensembl_ID | Gene | bp | Ensembl_ID | Gene | bp | Ensembl_ID | Gene | bp |
| ENSG00000230778 | ANKRD63 | 186 | ENSG00000105327 | BBC3 | 186 | ENSG00000241794 | SPRR2A | 196 |
| ENSG00000170128 | GPR25 | 93 | ENSG00000167566 | NCKAP5L | 93 | ENSG00000169397 | RNASE3 | 180 |
| ENSG00000183072 | NKX2-5 | 96 | ENSG00000141542 | RAB40B | 80 | ENSG00000169397 | RNASE3 | 180 |
| ENSG00000116990 | MYCL1 | 190 | ENSG00000187713 | TMEM203 | 85 | ENSG00000150269 | OR5M9 | 96 |
| ENSG00000235098 | RP4-758J18.6 | 187 | ENSG00000124216 | SNAI1 | 82 | ENSG00000155926 | SLA | 165 |
| ENSG00000115138 | POMC | 175 | ENSG00000169733 | RFNG | 79 | ENSG00000221819 | C16orf3 | 91 |
| ENSG00000107859 | PITX3 | 70 | ENSG00000142632 | ARHGEF19 | 79 | ENSG00000206102 | KRTAP19-8 | 63 |
| ENSG00000160972 | PPP1R16A | 174 | ENSG00000143416 | SELENBP1 | 84 | ENSG00000187475 | HIST1H1T | 72 |
| ENSG00000122136 | OBP2A | 173 | ENSG00000156413 | FUT6 | 193 | ENSG00000164379 | FOXQ1 | 71 |
| ENSG00000182095 | TNRC18 | 184 | ENSG00000174407 | C20orf166 | 170 | ENSG00000186047 | DLEU7 | 182 |
| ENSG00000149435 | GGTLC1 | 184 | ENSG00000212935 | KRTAP10-3 | 76 | ENSG00000140105 | WARS | 168 |
| ENSG00000177685 | EFCAB4A | 167 | ENSG00000130590 | SAMD10 | 96 | ENSG00000212127 | TAS2R14 | 65 |
| ENSG00000180155 | LYNX1 | 88 | ENSG00000092096 | SLC22A17 | 68 | ENSG00000204957 | AC006486.1 | 61 |
| ENSG00000162066 | AMDHD2 | 200 | ENSG00000054148 | PHPT1 | 93 | ENSG00000181518 | OR8D4 | 91 |
| ENSG00000255568 | NCRNA00257 | 184 | ENSG00000188095 | MESP2 | 167 | ENSG0000022670 | AL161915.1 | 64 |
| ENSG00000132329 | RAMP1 | 170 | ENSG00000175756 | AURKAIP1 | 162 | ENSG00000170465 | KRT6C | 167 |
| ENSG00000205143 | ARID3C | 199 | ENSG00000214819 | CDRT15L2 | 171 | ENSG00000170923 | OR7G2 | 71 |
| ENSG00000108785 | HSD17B1P1 | 167 | ENSG00000154016 | GRAP | 192 | ENSG00000248835 | AL357673.1 | 62 |
| ENSG00000087077 | TRIP6 | 73 | ENSG00000171223 | JUNB | 71 | ENSG00000107779 | BMPR1A | 164 |
| ENSG00000184601 | C14orf180 | 186 | ENSG00000108774 | RAB5C | 192 | ENSG00000169062 | UPF3A | 192 |
| ENSG00000178412 | AC068473.1 | 165 | ENSG00000186980 | KRTAP23-1 | 71 | ENSG00000169067 | ACTBL2 | 65 |
| ENSG00000131650 | KREMEN2 | 182 | ENSG00000214655 | KIAA0913 | 175 | ENSG00000008324 | SS18L2 | 163 |
| ENSG00000171471 | MAP1LC3B2 | 179 | ENSG00000236939 | C8orf56 | 198 | ENSG00000137080 | IFNA21 | 63 |
| ENSG00000101945 | SUV39H1 | 75 | ENSG00000049089 | COL9A2 | 174 | ENSG00000170605 | OR9K2 | 61 |
| ENSG00000001630 | CYP51A1 | 190 | ENSG00000099834 | CDHR5 | 167 | ENSG00000176281 | OR4K5 | 71 |
| ENSG00000198258 | UBL5 | 178 | ENSG00000144567 | FAM134A | 200 | ENSG00000214753 | HNRNPUL2 | 161 |
| ENSG00000187642 | Clorf170 | 89 | ENSG00000186193 | C9orf140 | 200 | ENSG00000106477 | TSGA14 | 192 |
| ENSG00000101198 | NKAIN4 | 80 | ENSG00000186844 | LCE1A | 173 | ENSG00000070831 | CDC42 | 164 |
| ENSG00000124449 | IRGC | 99 | ENSG00000064205 | WISP2 | 179 | ENSG00000197927 | C2orf27A | 175 |
| ENSG00000103024 | NME3 | 161 | ENSG00000162975 | KCNF1 | 71 | ENSG00000197927 | C2orf27A | 175 |
| ENSG00000003137 | CYP26B1 | 177 | ENSG00000175063 | UBE2C | 197 | ENSG00000169214 | OR6F1 | 94 |
| ENSG00000103266 | STUB1 | 172 | ENSG00000170935 | NCBP2L | 61 | ENSG00000221880 | KRTAP1-3 | 87 |
| ENSG00000162073 | PAQR4 | 97 | ENSG00000203863 | AL079342.1 | 62 | ENSG00000119669 | IRF2BPL | 98 |
| ENSG00000173457 | PPP1R14B | 187 | ENSG00000164900 | GBX1 | 173 | ENSG00000173402 | DAG1 | 194 |
| ENSG00000143258 | USP21 | 185 | ENSG00000142409 | ZNF787 | 172 | ENSG00000185899 | TAS2R60 | 63 |
| ENSG00000131037 | EPS8L1 | 84 | ENSG00000244623 | OR2AE1 | 881 | ENSG00000116489 | CAPZA1 | 169 |
| ENSG00000197723 | HSPB9 | 65 | ENSG00000186440 | OR6P1 | 88 | ENSG00000179528 | LBX2 | 164 |
| ENSG00000090971 | NAT14 | 200 | ENSG00000184009 | ACTG1 | 191 | ENSG00000212899 | KRTAP3-3 | 96 |
| ENSG00000163040 | CCDC74A | 200 | ENSG00000243811 | APOBEC3D | 164 | ENSG00000092199 | HNRNPC | 180 |
| ENSG00000106009 | BRAT1 | 78 | ENSG00000197837 | HIST4H4 | 76 | ENSG00000008988 | RPS20 | 168 |
| ENSG00000120913 | PDLIM2 | 78 | ENSG00000681241 | OR1F1 | 98 | ENSG00000143742 | SRP9 | 171 |
| ENSG00000100162 | CENPM | 196 | ENSG00000174599 | TRAM1L1 | 66 | ENSG00000178567 | EPM2AIP1 | 86 |
| ENSG00000139631 | CSAD | 96 | ENSG00000170948 | MBD3L1 | 71 | ENSG00000206260 | PRR23A | 86 |
| ENSG00000198892 | SHISA4 | 180 | ENSG00000188277 | Cl5orf62 | 67 | ENSG00000255622 | AC005754.1 | 81 |
| ENSG00000197540 | GZMM | 66 | ENSG00000228919 | AC097381.1 | 61 | ENSG00000184635 | ZNF93 | 183 |
| ENSG00000188997 | KCTD21 | 66 | ENSG00000184557 | SOCS3 | 174 | ENSG00000253459 | AL139099.1 | 68 |
| ENSG00000161714 | PLCD3 | 94 | ENSG00000173110 | HSPA6 | 197 | ENSG00000074201 | CLNS1A | 199 |
| ENSG00000115317 | HTRA2 | 94 | ENSG00000189159 | HN1 | 170 | ENSG00000114503 | NCBP2 | 195 |
| ENSG00000105085 | MED26 | 96 | ENSG00000176893 | OR51G2 | 82 | ENSG00000244537 | KRTAP4-2 | 182 |
| ENSG00000205220 | PSMB10 | 171 | ENSG00000154165 | GPR15 | 61 | ENSG00000250733 | C8orf17 | 82 |

### Example 3

### Target Enrichment of Multiplexed Panel Assays in Droplets vs. in Bulk

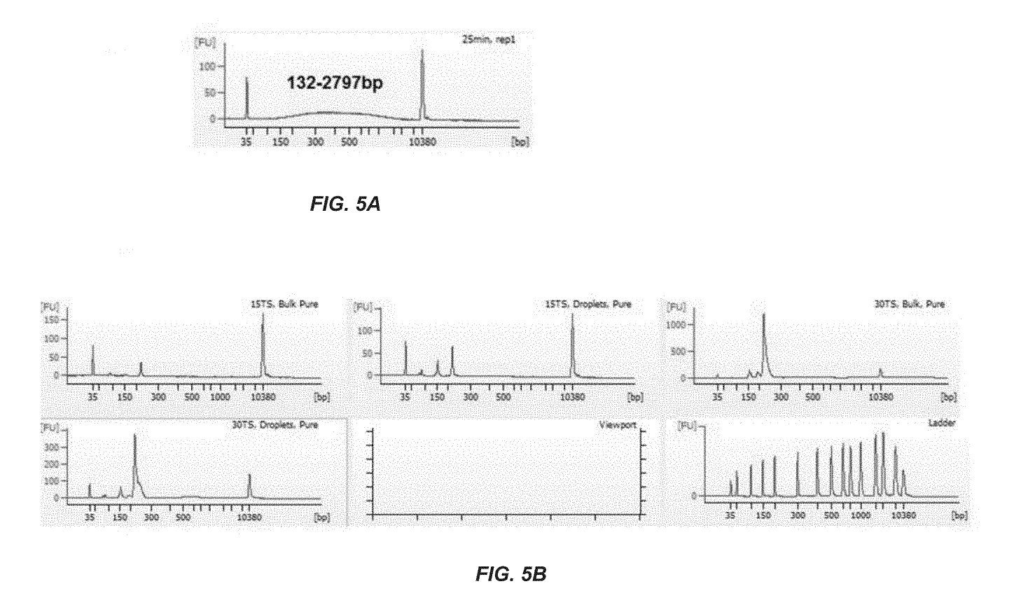

Target enrichment was performed for a 50-plex cancer panel using a target-specific, then nested PCR library construction as described in Example 1 above with the following modifications: A fragmented sample with a size districtuion of 132-2797 bp was used (see FIG. 5A). Two trials of target-specific amplification were performed (one with 15 cycles of target-specific PCR, one with 30 cycles of target-specific PCR) with a 45° C. annealing temperature. Droplet breaking was accomplished using chloroform. For sequencing, 10% PhiX or 50% PhiX was included as a spike-in for increasing the diversity of sequence reads.

As shown in FIG. 5B, the amplicons subject to 15 or 30 cycles of target-specific PCR followed by 30 cycles of nested PCR and then 1× AMPure-purifications gave rise to high yields of what appear to be amplicon libraries. For both bulk and droplets, the concentrations were significantly higher for the nested PCR derived from 30 cycles of target-specific PCR relative to 15 cycles of target-specific PCR.

### Example 4

### Target Enrichment of Multiplexed Panel Assays Using Different Target-Specific Amplification Master Mix Formulations

Target enrichment was performed for a 50-plex cancer panel using a target-specific, then nested PCR library construction as described in Example 3 above with the following modifications. Two target-specific PCR mixes were tested: SsoAdvanced PreAmp Supermix without KCl added (for bulk PCR), and ddPCR Supermix no dUTP with 40 mM of KCl added (for droplet PCR). Target-specific amplification was performed for 30 cycles with a 55-45° C. annealing gradient for 4 min. For the nested PCR amplification, the annealing temperature was raised to 65° C. 15 cycles of nested PCR amplification were performed.

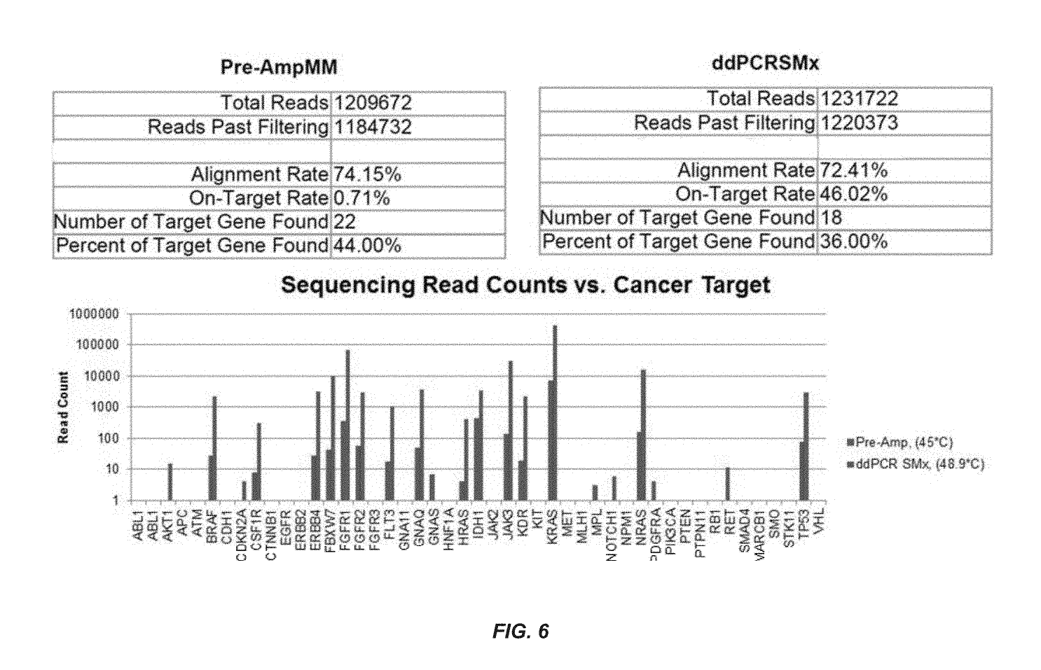

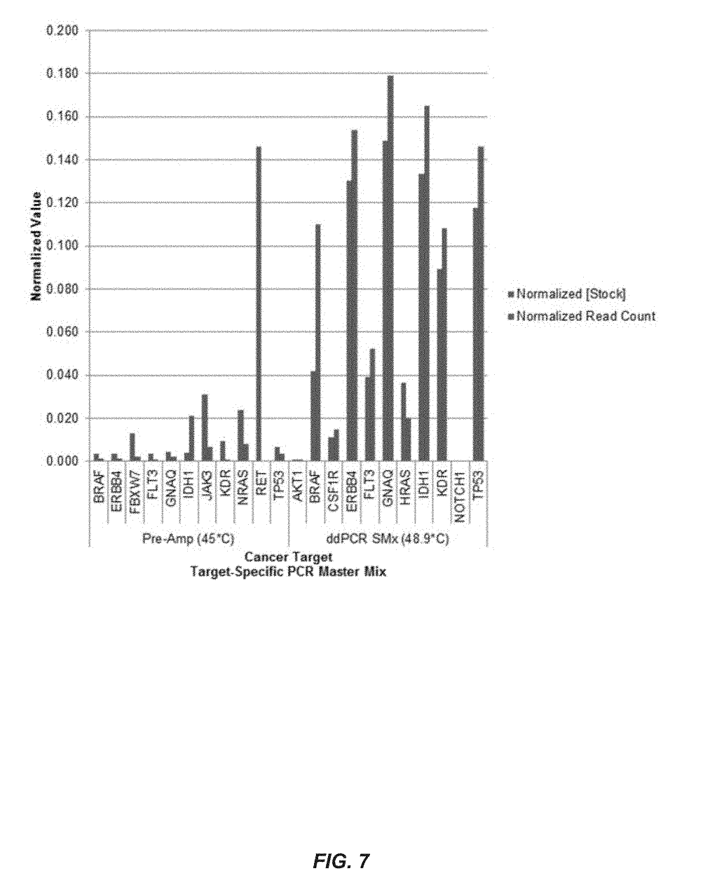

As shown in FIG. 6, target-specific PCR in droplets with the ddPCR Supermix yielded a significantly higher on-target rate as compared to PCR in bulk with the PreAmp Supermix (46.02% vs. 0.71%). There was a master-mix dependent preferential amplification of some targets over others (FIG. 6). The normalized correlation analysis shown in FIG. 7 demonstrates that significantly higher amplicon yields were obtained from ddPCR Supermix than from the PreAmp master mix.

### Example 5

### Target Enrichment of Multiplexed Panel Assays in Droplets or in Bulk

Target enrichment was performed for a 50-plex cancer panel and a 48-plex cancer panel in bulk or in droplets using a target-specific, then nested PCR library construction as described in Example 4 above with the following modifications. Target-specific amplification was performed for 30 cycles at a 45° C. annealing temperature for 4 min. For the 48-plex, the cancer targets KRAS and IDH1 were excluded by excluding KRAS and IDH1 primers from the target-specific amplification master mixes. The target-specific amplification master mixes ABI Gene Expression and ABI Genotyping were also tested. For the nested PCR amplification step, 30 cycles of nested PCR amplification were performed.

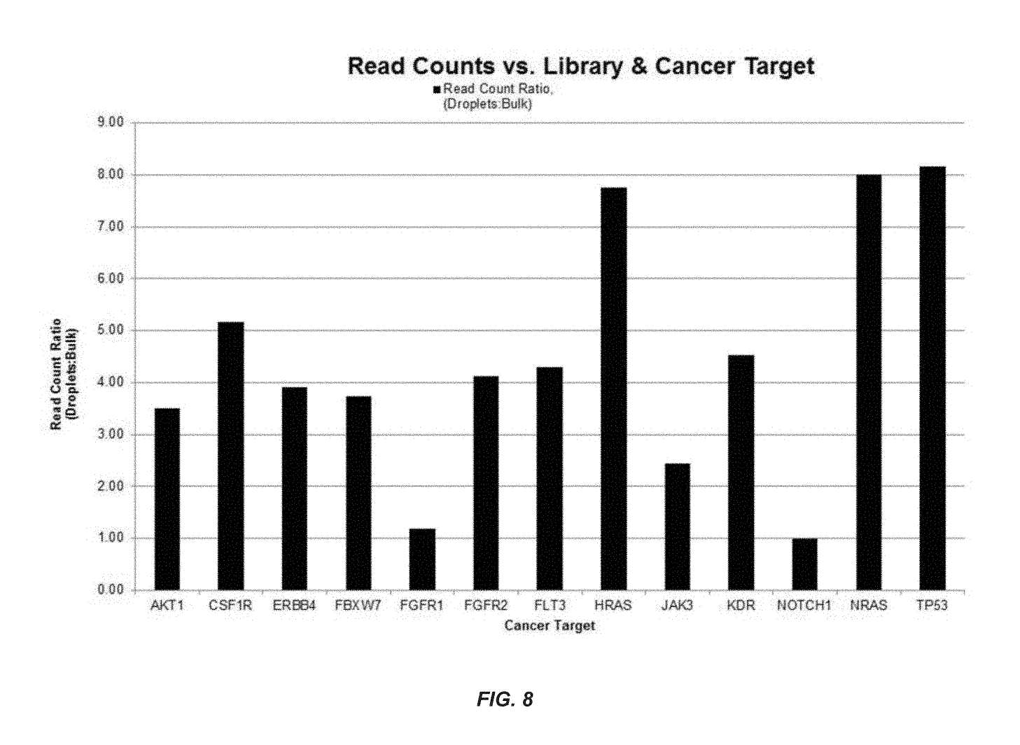

FIG. 8 shows a ratio of sequencing read counts derived from library 8 (generated by target-specific PCR in droplets using ddPCR supermix) vs. library 9 (generated by target-specific PCR in bulk using ddPCR supermix) on the y-axis. The x-axis shows cancer targets in the 48-plex. The values for the ratios in FIG. 8 are all greater than 1, indicating that there is more sequencing data for the targets derived from droplet amplification as compared to targets derived from bulk amplification. Additionally, in many instances there was an approximately 4-8 fold increased yield of amplicons recovered from droplets relative to those in bulk. This demonstrates the enhanced competition of PCR amplicons with poor efficiency as isolated in droplets relative to in bulk.

### Example 6

### Target Enrichment of Multiplexed Panel Assays in Droplets

Target enrichment was performed for a 48-plex cancer panel in bulk or in droplets using a target-specific, then nested PCR library construction as described in Example 5 above with the following modifications. A new source of human genomic DNA was used (BioChain Institute, Inc., Newark, Calif.), and was fragmented using a fragmentase for 20 minutes to an average size of 865 bp (distribution of 152-6750 bp). For target-specific PCR, ddPCR Supermix was tested in bulk vs. droplets with or without a 40 mM KCl spike-in. Target-specific amplification was performed for 30 cycles at a 45° C. annealing temperature for 1 min. Nested PCR amplification was performed using the P5 RD1 primer and the P7 Index “version 2” primers shown in Table 5 below. These primers use adapter indexes that are the reverse complements of the Illumina TruSeq indexes in BaseSpace for ease of analyzing the sequencing data obtained.

The JMP statistical SAS software program's Prediction Profiler was used to maximize the un-normalized read count (per Bio-Rad TruSeq ddPCR concentration determinations on a per-library basis) based on the inputs of PCR annealing time and cancer target. For determining un-normalized read count, each library was loaded onto the sequencer on a normalized basis to equimolar and the normalization was mathematically reversed to account for the relative yields of the libraries from the library construction protocol. A mild slope was found between 1 and 4 minute annealing times, meaning that this factor was relatively unimportant in yielding maximal un-normalized read counts. The data for the cancer targets had many peaks with sharp slopes, demonstrating that success in evening out sequence coverage is target-dependent.

The data provided herein suggests that even sequencing coverage can be enhanced by optimizing conditions such as the master mix formulation and PCR conditions. Additionally, the JMP Prediction Profiler and Interaction Profile can be used to demonstrate optimal conditions for obtaining a desired output (e.g., for maximizing reads).

TABLE 5
P7 Index RD2 Primers

| Primer | | SEQ |
|---|---|---|
| Name | Sequence | ID NO |
| P7 Index1 | CAAGCAGAAGACGGCATACGAGATCGTGATGT | 113 |
| RD2 v2 | GACTGGAGTTCAGACGTGTGCTCTTCCGATCT | |
| P7 Index2 | CAAGCAGAAGACGGCATACGAGATACATCGGT | 114 |
| RD2 v2 | GACTGGAGTTCAGACGTGTGCTCTTCCGATCT | |
| P7 Index3 | CAAGCAGAAGACGGCATACGAGATGCCTAAGT | 115 |
| RD2 v2 | GACTGGAGTTCAGACGTGTGCTCTTCCGATCT | |
| P7 Index4 | CAAGCAGAAGACGGCATACGAGATTGGTCAGT | 116 |
| RD2 v2 | GACTGGAGTTCAGACGTGTGCTCTTCCGATCT | |
| P7 Index5 | CAAGCAGAAGACGGCATACGAGATCACTGTGT | 117 |
| RD2 v2 | GACTGGAGTTCAGACGTGTGCTCTTCCGATCT | |
| P7 Index6 | CAAGCAGAAGACGGCATACGAGATATTGGCGT | 118 |
| RD2 v2 | GACTGGAGTTCAGACGTGTGCTCTTCCGATCT | |
| P7 Index7 | CAAGCAGAAGACGGCATACGAGATGATCTGGT | 119 |
| RD2 v2 | GACTGGAGTTCAGACGTGTGCTCTTCCGATCT | |
| P7 Index8 | CAAGCAGAAGACGGCATACGAGATTCAAGTGT | 120 |
| RD2 v2 | GACTGGAGTTCAGACGTGTGCTCTTCCGATCT | |
| P7 Index9 | CAAGCAGAAGACGGCATACGAGATCTGATCGT | 121 |
| RD2 v2 | GACTGGAGTTCAGACGTGTGCTCTTCCGATCT | |
| P7 Index10 | CAAGCAGAAGACGGCATACGAGATAAGCTAGT | 122 |
| RD2 v2 | GACTGGAGTTCAGACGTGTGCTCTTCCGATCT | |
| P7 Index11 | CAAGCAGAAGACGGCATACGAGATGTAGCCGT | 123 |
| RD2 v2 | GACTGGAGTTCAGACGTGTGCTCTTCCGATCT | |
| P7 Index12 | CAAGCAGAAGACGGCATACGAGATTACAAGGT | 124 |
| RD2 v2 | GACTGGAGTTCAGACGTGTGCTCTTCCGATCT | |
| P7 Index13 | CAAGCAGAAGACGGCATACGAGATTTGACTGT | 125 |
| RD2 v2 | GACTGGAGTTCAGACGTGTGCTCTTCCGATCT | |
| P7 Index14 | CAAGCAGAAGACGGCATACGAGATGGAACTGT | 126 |
| RD2 v2 | GACTGGAGTTCAGACGTGTGCTCTTCCGATCT | |
| P7 Index15 | CAAGCAGAAGACGGCATACGAGATTGACATGT | 127 |
| RD2 v2 | GACTGGAGTTCAGACGTGTGCTCTTCCGATCT | |
| P7 Index16 | CAAGCAGAAGACGGCATACGAGATGGACGGGT | 128 |
| RD2 v2 | GACTGGAGTTCAGACGTGTGCTCTTCCGATCT | |
| P7 Index18 | CAAGCAGAAGACGGCATACGAGATGCGGACGT | 129 |
| RD2 v2 | GACTGGAGTTCAGACGTGTGCTCTTCCGATCT | |
| P7 Index19 | CAAGCAGAAGACGGCATACGAGATTTTCACGT | 130 |
| RD2 v2 | GACTGGAGTTCAGACGTGTGCTCTTCCGATCT | |
| P7 Index20 | CAAGCAGAAGACGGCATACGAGATGGCCACGT | 131 |
| RD2 v2 | GACTGGAGTTCAGACGTGTGCTCTTCCGATCT | |
| P7 Index21 | CAAGCAGAAGACGGCATACGAGATCGAAACGT | 132 |
| RD2 v2 | GACTGGAGTTCAGACGTGTGCTCTTCCGATCT | |
| P7 Index22 | CAAGCAGAAGACGGCATACGAGATCGTACGGT | 133 |
| RD2 v2 | GACTGGAGTTCAGACGTGTGCTCTTCCGATCT | |
| P7 Index23 | CAAGCAGAAGACGGCATACGAGATCCACTCGT | 134 |
| RD2 v2 | GACTGGAGTTCAGACGTGTGCTCTTCCGATCT | |
| P7 Index25 | CAAGCAGAAGACGGCATACGAGATATCAGTGT | 135 |
| RD3 v2 | GACTGGAGTTCAGACGTGTGCTCTTCCGATCT | |
| P7 Index27 | CAAGCAGAAGACGGCATACGAGATAGGAATGT | 136 |
| RD4 v2 | GACTGGAGTTCAGACGTGTGCTCTTCCGATCT | |

INFORMAL SEQUENCE LISTING

P5 adapter sequence
SEQ ID NO: 1
5′-AAT GAT ACG GCG ACC ACC GAG ATC TAC ACT CTT TCC
CTA CAC GAC GCT CTT CCG ATC T-3′
P5 universal adapter sequence
SEQ ID NO: 2
AATGATACGGCGACCACCGAGATCT
P5 index adapter sequence
SEQ ID NO: 3
5′-AAT GAT ACG GCG ACC ACC GAG ATC TNN NNN NAC ACT
CTT TCC CTA CAC GAC GCT CTT CCG ATC T-3′
P7 adapter sequence
SEQ ID NO: 4
5-CAA GCA GAA GAC GGC ATA CGA GAT GTG ACT GGA GTT
CAG ACG TGT GCT CTT CCG ATC T-3′
P7 universal adapter sequence
SEQ ID NO: 5
CAAGCAGAAGACGGCATACGAGAT
P7 index adapter sequence
SEQ ID NO: 6
5-CAA GCA GAA GAC GGC ATA CGA GAT NNN NNN GTG ACT
GGA GTT CAG ACG TGT GCT CTT CCG ATC T-3′
Partial P5 adapter sequence
SEQ ID NO: 7
5′-ACACTCTTTCCCTACACGACGCTCTTCCGATCT-3′
Partial P7 adapter sequence
SEQ ID NO: 8
5′-TCAGACGTGTGCTCTTCCGATCT-3′
SEQ ID NOs: 9-58- Partial P7 + forward gene-
specific primer sequences (Table 1)
SEQ ID NOs: 59-108- Partial P5 + reverse gene-
specific primer sequences (Table 2)
Index 1 Read adapter sequence
SEQ ID NO: 109
5′-CAAGCAGAAGACGGCATACGAGAT[i7]GTCTCGTGGGCTCGG-3′
Index 2 Read adapter sequence
SEQ ID NO: 110
5′-AATGATACGGCGACCACCGAGATCTACAC[i5]TCGTCGGCAGCG
TC-3′
SEQ ID NO: 111- P7 Index6 RD2 adapter sequences
SEQ ID NO: 112- P7 Index12 RD2 adapter sequences
SEQ ID NOs: 113- 136-P7 Index RD2 version 2 adapter
sequences

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of preparing a target gene-enriched library, the method comprising:
(a) providing a plurality of polynucleotide fragments;
(b) partitioning the polynucleotide fragments into a plurality of partitions, wherein each partition further comprises a plurality of primer pairs, each primer pair comprising a forward primer and a reverse primer for amplifying a target gene, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence;
(c) amplifying a target gene sequence of a polynucleotide fragment in a partition with one of the primer pairs in the partition, thereby generating an amplicon comprising the target gene sequence flanked on the 5′ end by the portion of the first adapter sequence and flanked on the 3′ end by the portion of the second adapter sequence;
(d) purifying the amplicon; and
(e) amplifying the amplicon using a first amplicon primer comprising at least a portion of the first adapter sequence and a second amplicon primer comprising at least a portion of the second adapter sequence.

2. The method of claim 1, wherein the polynucleotide fragments are genomic DNA fragments.

3. The method of claim 1, wherein the polynucleotide fragments are at least about 100 nucleotides in length.

4. (canceled)

5. The method of claim 1, wherein in the partitioning step (b), each partition comprises at least 50 primer pairs.

6. (canceled)

7. The method of claim 1, wherein a target gene for amplification is a gene having a rare mutation.

8. The method of claim 1, wherein (i) the first adapter sequence is a P7 adapter sequence and the second adapter sequence is a P5 adapter sequence; or (ii) the first adapter sequence is a P5 adapter sequence and the second adapter sequence is a P7 adapter sequence.

9. The method of claim 8, wherein the first adapter sequence is a P7 adapter sequence having at least 70% identity to SEQ ID NO:4.

10. The method of claim 1, wherein the forward primer comprising a portion of the first adapter sequence comprises at least 20 contiguous nucleotides of the first adapter sequence.

11. The method of claim 10, wherein the portion of the first adapter sequence has at least 70% identity to SEQ ID NO:8.

12. The method of claim 8, wherein the second adapter sequence is a P5 adapter sequence having at least 70% identity to SEQ ID NO:1.

13. The method of claim 1, wherein the reverse primer comprising a portion of the second adapter sequence comprises at least 20 contiguous nucleotides of the second adapter sequence.

14. The method of claim 13, wherein the portion of the second adapter sequence has at least 70% identity to SEQ ID NO:7.

15. The method of claim 1, wherein the first adapter sequence and/or the second adapter sequence comprises a barcode sequence.

16. (canceled)

17. The method of claim 1, wherein the partitions are droplets.

18-19. (canceled)

20. The method of claim 1, wherein the partitions comprise an average of about 0.1 to about 10 targets per droplet.

21-24. (canceled)

25. The method of claim 1, wherein the amplifying step (e) comprises at least 10 cycles of amplification.

26-27. (canceled)

28. The method of claim 1, wherein following the amplifying step (e), the method further comprises sequencing at least one amplicon.

29. A library of amplicons generated according to the method of claim 1.

30. A kit comprising:
(a) a first composition for partitioning into a plurality of partitions, wherein the composition comprises a plurality of primer pairs, each primer pair comprising a forward primer and a reverse primer for amplifying a target gene, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence; and
(b) a second composition comprising a first primer and a second primer, wherein the first primer comprises the first adapter sequence and the second primer comprises the second adapter sequence.

31. A method for detecting a plurality of targets in a biological sample, the method comprising:
(a) obtaining a plurality of polynucleotide fragments from the biological sample;
(b) partitioning the polynucleotide fragments into a plurality of partitions, wherein each partition further comprises a plurality of primer pairs, each primer pair comprising a forward primer and a reverse primer for amplifying a target gene, wherein the forward primer comprises (i) a polynucleotide sequence that comprises a portion of a first adapter sequence and (ii) a target gene-specific forward primer sequence, and wherein the reverse primer comprises (i) a polynucleotide sequence that comprises a portion of a second adapter sequence and (ii) a target gene-specific reverse primer sequence;
(c) amplifying a target gene sequence of a polynucleotide fragment in a partition with one of the primer pairs in the partition, thereby generating an amplicon comprising the target gene sequence flanked on the 5′ end by the portion of the first adapter sequence and flanked on the 3′ end by the portion of the second adapter sequence;
(d) purifying the amplicon;
(e) amplifying the amplicon using a first primer comprising the first adapter sequence and a second primer comprising the second adapter sequence; and
detecting a plurality of amplicons from the amplifying step (e).

32-33. (canceled)
